(19) United States
(12) Patent Application Publication
(10) Pub. No.: US 2024/0293431 A1
(43) Pub. Date: Sep. 5, 2024

(54) COMPOSITIONS AND METHODS FOR THE TREATMENT AND/OR PREVENTION OF ALZHEIMER'S DISEASE

(71) Applicant: UNIVERSITY OF SOUTH FLORIDA, Tampa, FL (US)

(72) Inventor: Chuanhai Cao, Tampa, FL (US)

(21) Appl. No.: 18/593,708

(22) Filed: Mar. 1, 2024

Related U.S. Application Data
(60) Provisional application No. 63/488,236, filed on Mar. 3, 2023.

(51) Int. Cl. A61K 31/00; A61K 9/107; A61K 31/4045; A61P 25/28

(52) U.S. Cl. CPC A61K 31/658; A61K 9/1075; A61K 31/4045; A61P 25/28

(57) ABSTRACT

Methods and compositions for treating and/or preventing Alzheimer's disease (AD), and/or for reducing one or more amyloid beta peptides and/or phosphorylated tau (p-tau) in the brain of a subject. The methods can include administering a composition comprising melatonin and cannabidiol (CBD).

### CROSS-REFERENCE TO RELATED APPLICATIONS

This application claim priority to U.S. Application No. 63/488,236, filed on Mar. 3, 2023, the entire contents of which are hereby incorporated by reference.

### STATEMENT REGARDING FEDERALLY SPONSORED RESEARCH OR DEVELOPMENT

N/A

### BACKGROUND

Alzheimer's disease (AD) is the most prevalent neurodegenerative disease worldwide, characterized by memory loss and progressive cognitive disability. Although AD is a disease with complex etiology, it has two main hallmarks: extracellular deposits of the amyloid-β peptide (Aβ) and intracellular neurofibrillary tangles (NFT) composed of hyperphosphorylated tau protein (p-tau). These deposits mainly occur in the neocortex, hippocampus, and other subcortical regions of the brain that are pivotal in carrying out cognitive function.

### SUMMARY

In one aspect, a method of treating and/or preventing AD is provided. The method can include administering a therapeutically effective amount of a composition to a subject in need thereof, where the composition includes cannabidiol (CBD) or a pharmaceutically acceptable salt thereof, and melatonin or a pharmaceutically acceptable salt thereof.

In another aspect, a method of reducing one or more amyloid beta peptides and/or p-tau in the brain of a subject is provided. The method can include administering a therapeutically effective amount of a composition to a subject in need thereof, where the composition includes cannabidiol (CBD) or a pharmaceutically acceptable salt thereof, and melatonin or a pharmaceutically acceptable salt thereof.

In yet another aspect, composition in the form of an emulsion is provided. The composition can include cannabidiol (CBD) or a pharmaceutically acceptable salt thereof and melatonin or a pharmaceutically acceptable salt thereof.

### BRIEF DESCRIPTION OF THE FIGURES

FIGS. 1A-1B. Evaluation of the spatial memory of aged C57BL/6J and APP/PS1 mice using RAWM test; pre-treatment behavior study. The cognitive performances of 12-month-old NTG and TG mice were presented as trials of the number of errors (FIG. 1A) and latency (FIG. 1B). Both parameters demonstrate the difference in cognitive performance at the learning phase-trial 1 (T1) and at the memory phase-trial 5 (T5). The pre-treatment RAWM test demonstrated that the learning memory of the TG mice was significantly impaired as compared with the NTG mice during the last trial of the test (T5). The values are expressed as means±SEM for NTG mice (n=11) and TG mice (n=20). Statistical analysis of the number of errors and escape latencies was performed using a two-way ANOVA followed by a multiple comparison test. **p<0.01 indicates a significant difference between T1 and T5 for each group.

FIGS. 2A-2B. Evaluation of the spatial memory of APP/PS1 after three months of MIC or MC intranasal treatment; post-treatment behavior study. The number of errors (FIG. 2A) and latency (FIG. 2B) of the post-treatment study represent the difference in cognitive performance of 15-month-old continuously MIC or MC-treated TG mice from the TG and NTG groups. T1 represents trial 1 of the learning phase, and T5 represents trial 5 of the memory phase. The behavior test data showed that 3 months of daily intranasal treatment with MIC or MC nanoformulation significantly reduced (FIG. 2A) the number of errors and approached the NTG mouse group data. The RAWM study of 15-month-old mice revealed a significant reduction in (FIG. 2B) latency in the cognitive performance of MC-treated mice. The TG control group demonstrated steady memory decline, and the treatment groups show significant cognitive improvement. The data is expressed as the mean±SEM of n mice per group (NTG group n=11, TG group n=7; MIC group n=6; MC group n=7). SEM is denoted by the error bars. Latency and the number of errors were analyzed using a two-way ANOVA followed by a multiple comparison test. The NTG, TG, MIC-treated, and MC-treated TG mice were compared (*p<0.05, **p<0.01, and ***p<0.001).

FIGS. 3A-3B. The assessment of changes in Aβ levels in the plasma and brain tissue of APP/PS1 mice after the three-month intranasal treatment with the MIC or the MC nanoformulations. (FIG. 3A) Plasma was analyzed by the sandwich ELISA for Aβ assay to measure concentrations of secreted Aβ species, and the Aβ42/40 ratio was determined. Plasma samples were collected from the APP/PS1 mice at the beginning, in the middle, and after the intranasal treatment course. A significant change in Aβ42/40 ratio was detected in MIC and MC treatment group samples. The data are shown as mean±SEM (n=6 for each group). Statistical analysis was performed using a two-way ANOVA followed by a multiple comparison test. (FIG. 3B) Soluble Aβ40 and Aβ42 peptides, and Aβ oligomer levels in the hippocampal tissue were evaluated by Aβ sandwich ELISA. MIC and MC treatments induce a trend toward a decrease of Aβ40 and Aβ oligomer in the brain; however, no significant difference was found among the study groups. A considerable reduction of hippocampal Aβ42 peptide was detected in MC-treated mice samples. The data are presented as mean±SEM (n=6). A one-way ANOVA was used to analyze the data, followed by a Tukey post hoc multiple comparison test. The NTG, TG, MIC-treated, and MC-treated TG mice were compared (*p<0.05, **p<0.01, and ***p<0.001).

FIGS. 4A-4D. Immunoblot analysis of molecular markers associated with the neuropathologic changes in AD. (FIG. 4A) Representative Western blot analysis of p-tau, tau, β-actin, GSK3β, p-GSK3β in the brain tissue of NTG, TG, MIC, and MC mice. (FIG. 4B) Relative protein expression levels of tau and p-tau were normalized to β-actin. A significant decrease in the expression of p-tau was observed in the brains of nanoformulation-treated APP/PS1 mice. MC-treated mice demonstrated upregulated tau expression in comparison to all mice groups. (FIG. 4C) Relative protein expression levels of GSK3β and p-GSK3β were normalized to β-actin. A stimulation of significant increase in p-GSK3β production was discovered after prolonging daily nasal treatment with MC formulation. (FIG. 4D) Comparison of p-tau vs tau and p-GSK3β vs GSK3β rates in different groups. Long-term MC-treated mice had significantly lower p-tau/tau ratio in comparison to the TG group, indicating that the melatonin-CBD-based nanoformulation has a beneficial effect on AD neuropathologies. A remarkable increase in the p-GSK3β/GSK3β ratio was associated with nasal treatments involving both nanoformulations. The data are expressed as mean±SEM (n=6 for the NTG, TG, and MIC groups and 7 for the MC group). SEM is denoted by the error bars. Using a one-way ANOVA followed by a Tukey post hoc multiple comparison test, * p<0.05, **p<0.01, and ***p<0.001 were calculated for the NTG, TG, MIC, and MC-treated mouse groups.

FIGS. 5A-5B. Assessment of the MIC and MC nasal treatment effect on the mitochondrial functions in the hippocampus using blotting analysis. The brain tissue samples were collected from the TG mice after the 3-month MIC or MC nasal treatment and from the TG and NTG control mice. (FIG. 5A) Representative immunoblots of the expression of TFAM, β-actin, CKMT1, and MFF proteins. (FIG. 5B) Relative protein expression levels of TFAM, CKMT1, and MFF normalized to β-actin. Densitometric analysis of blot results demonstrated a significant difference in the expression of MFF and TFAM proteins in the brains of TG and MIC-treated mice in comparison to NTG mice. Significant difference in CKMT1 expression was found between all TG animals and NTG control groups. The data are expressed as mean±SEM (n=6 for the NTG group; n=6 for the TG group; n=6 for the TG MIC-treated group; and n=7 for the TG MC-treated group). SEM is denoted by error bars. *p<0.05, **p<0.01, and ***p<0.001 were compared among all groups by a one-way ANOVA followed by a Tukey post hoc multiple comparison test.

FIGS. 6A-6D. Changes in mitochondrial fission/fusion and mitophagy proteins following continuous intranasal administration of the MIC or MC nanoformulations. (FIG. 6A) Representative immunoblot analysis of Fis1, Mfn2, β-actin, Mfn1, Drp1, Opa1, Parkin, and Pink1 in the brain tissue of MIC-treated, MC-treated, TG, and NTG control mice. The immunoblotting quantification of Opa1, Mfn1, Mfn2 (FIG. 6B), Drp1, Fis1 (FIG. 6C), and Pink1, and Parkin (FIG. 6D). (FIG. 6B) No significant difference in all fusion proteins (Opa1, Mfn1, and Mfn2) was found between the NTG and TG groups. Upregulation of fusion proteins was observed after a course of intranasal treatment with MIC or MC. (FIG. 6C) A significant increase in fission proteins (Drp1 and Fis1) was detected in TG mice in comparison to NTG. A noticeable impact on Drp1 toward downregulation and Fis1 toward upregulation was provided by long-term MIC nasal treatment. Importantly, MC treatment did not have that significant impact on fission proteins in comparison to MIC. (FIG. 6D) A significant increase in the mitophagy protein Parkin expression was detected in TG mice in comparison to NTG. MC nanoformulations significantly upregulated Pink1 and downregulated Parkin expression. The data are presented as mean SEM (n=6 for the NTG group; n=6 for the TG group; n=6 for the TG MIC-treated group; and n=7 for the TG MC-treated group). SEM is denoted by the error bars. Using a one-way ANOVA followed by a Tukey post hoc multiple comparison test, * p<0.05, ** p<0.01, and *** p<0.001 were calculated for the NTG, TG, and treatment TG groups.

FIGS. 7A-7F. Immunohistochemical analysis of molecular markers associated with the AD neuropathologic profile in hippocampi. (FIGS. 7A-7C) Coronal slices of the left cerebral hemisphere from NTG, TG, MIC, and MC treated TG mice. Representative Aβ oligomer (FIG. 7A), Iba1 (FIG. 7B), and NeuN (FIG. 7C) staining images of brain sections. (FIGS. 7D-7F) Quantification of IHC staining of Aβ-oligomer (FIG. 7D), Iba1 (FIG. 7E), and NeuN (FIG. 7F). IHC results are presented as the percentage of Aβ oligomer, NeuN, and Iba1 stained positive tissue areas per field in the brain. No statistically significant differences in Aβ oligomer were found between MIC and TG control animal group. MC treatment induced a considerable decrease of neurotoxic Aβ isoform. A statistically significant difference in Iba1 was found between the MC-treated and TG animal groups. Thus, the MC-treatment group approached the Iba1 level of NTG mice. The MIC-treated mice had a significantly higher number of NeuN-positive cells than the TG control mice. The data are presented as mean±SEM (n=5-6). *p<0.05, **p<0.01, and ***p<0.001 compared among NTG, TG, MIC-treated, and MC-treated mice using a one-way ANOVA followed by a Tukey post hoc multiple comparison test.

### DETAILED DESCRIPTION

### Overview

The present disclosure relates to methods and compositions for the treatment and/or prevention of AD. As discussed above, AD is the most prevalent neurodegenerative disease worldwide, characterized by memory loss and progressive cognitive disability. Although AD is a disease with complex etiology, it has two main hallmarks: extracellular deposits of the amyloid-β peptide (Aβ) and intracellular neurofibrillary tangles (NFT) composed of hyperphosphorylated tau protein (p-tau). These deposits mainly occur in the neocortex, hippocampus, and other subcortical regions of the brain that are pivotal in carrying out cognitive function. While much emphasis has been given to the amyloid and tau proteins, it is evident that AD has a multifactorial etiology. Mitochondrial dysfunction, oxidative stress, insulin intolerance, and neurodegeneration are known to contribute to the pathogenesis of AD.

As of now, treatments targeting a single pathogenic factor of the disease, such as inhibition of Aβ peptide aggregation or tau phosphorylation, have yet to yield satisfactory results. Thus, a treatment that could affect multiple aspects of the disease pathogenesis would be the most beneficial approach in AD therapy. In other words, to halt disease progression, medications should interfere with all AD pathogenic events, which are responsible for all the clinical symptoms, including the formation of amyloid plaques and NFT, inflammation, oxidative damage, metabolism dysregulation, etc. Despite the wealth of studies on AD pathology and intense research efforts in the past few decades, no effective therapeutic approach has been translated into clinical application. Therefore, there is an utmost need for the development of new, more effective multitargeting therapeutics that can slow or delay the onset of AD and prevent the progression of the disease by targeting multiple AD-related pathological processes simultaneously. In various aspects, the present disclosure relates to meeting this need. For instance, in certain aspects, the methods disclosed herein can include administering a therapeutically effective amount of a composition that include CBD and melatonin.

### Definitions

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

### Peptides, Polypeptides, Proteins, and Synthesis Methods

As used herein, the terms “peptide,” “polypeptide,” and “protein,” refer to molecules comprising a chain a polymer of amino acid residues joined by amide linkages. The term “amino acid residue,” includes but is not limited to amino acid residues contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include nonstandard or unnatural amino acids. The term “amino acid residue” may include alpha-, beta-, gamma-, and delta-amino acids.

As used herein, a “peptide” is defined as a short polymer of amino acids, of a length typically of 20 or less amino acids, and more typically of a length of 12 or less amino acids (Garrett & Grisham, Biochemistry, 2nd edition, 1999, Brooks/Cole, 110). In some embodiments, a peptide as contemplated herein may include no more than about 2, 3, 4, 5, 6, 7, 8, 9, 10, 11, 12, 13, 14, 15, 16, 17, 18, 19, or 20 amino acids. A polypeptide, also referred to as a protein, is typically of length >100 amino acids (Garrett & Grisham, Biochemistry, 2nd edition, 1999, Brooks/Cole, 110).

### Compositions

In various aspects, the compositions disclosed herein can include cannabidiol (CBD) or a pharmaceutically acceptable salt thereof, and melatonin or a pharmaceutically acceptable salt thereof. In certain aspects, the compositions disclosed herein can include CBD or a pharmaceutically acceptable salt thereof, melatonin or a pharmaceutically acceptable salt thereof, and insulin or a pharmaceutically acceptable salt thereof.

In one or more aspects, the compositions disclosed herein can comprise or consist essentially of CBD or a pharmaceutically acceptable salt thereof, and melatonin or a pharmaceutically acceptable salt thereof. In various aspects, the compositions disclosed herein can comprise or consist essentially of CBD or a pharmaceutically acceptable salt thereof, melatonin or a pharmaceutically acceptable salt thereof, and insulin or a pharmaceutically acceptable salt thereof.

In certain aspects, the CBD can be present in the composition in an amount of about 300 milligrams (mg)/milliliter (ml) to about 1500 mg/ml, about 500 milligrams (mg)/milliliter (ml) to about 1200 mg/ml, or about 700 milligrams (mg)/milliliter (ml) to about 900 mg/ml. In one or more aspects, the CBD can be present in the composition in an amount of about 830 mg/ml.

In various aspects, the melatonin can be present in the composition in an amount of about 10 mg/ml to about 1000 mg/ml, about 50 mg/ml to about 500 mg/ml, or about 100 mg/ml to about 200 mg/ml. In certain aspects, the melatonin can be present in the compositions in an amount of about 167 mg/ml.

In certain aspects, the insulin can be present in the composition in an amount of about 1 Unit (U)/ml to about 100 U/ml, about 5 Units (U)/ml to about 70 U/ml, or about 10 Units (U)/ml to about 30 U/ml. In certain aspects, the insulin can be present in an amount of about 20 U/ml.

In various aspects, the composition can include one or more of an emulsifier, an oil, or an aqueous solvent. In one or more aspects, the aqueous solvent can be any aqueous solvent that is suitable for use with CBD, melatonin, and insulin. In certain aspects, the aqueous solvent can be water.

In certain aspects, the emulsifier can be any emulsifier that is suitable for use in the compositions and with the other components disclosed herein. In one aspect, the emulsifier is suitable for at least partly forming the composition into an emulsion and/or a nanoemulsion. In various aspects, the emulsifier can be lecithin.

In certain aspects, the oil can be any suitable oil for use with the compositions and/or other components disclosed herein. In certain aspects, the oil can comprise a medium chain triglyceride (MCT) oil. The MCT oil can be derived from any suitable source. In various aspects, the MCT oil can comprise a glycerol backbone with ester linkages to one or more carbon chains comprising about 4 carbon atoms to about 18 carbon atoms, or about 6 carbon atoms to about 12 carbon atoms.

In certain aspects, the compositions be an emulsion or formed into an emulsion. In one or more aspects, the emulsion can include an oil phase and an aqueous or water phase. In various aspects, the emulsion can be a nanoemulsion. The terms “nanoformulation” and “nanoemulsion” may be used interchangeably herein. A nanoformulation may contain an oil phase and a water phase. In some aspects, CBD can be dissolved in the oil phase of an emulsion. In the same or alternative aspects, insulin and/or melatonin can be dissolved in the water or aqueous phase of the emulsion.

In certain aspects, in the nanoemulsion, the oil phase and/or the aqueous phase can be present as droplets. In various aspects, the droplets can have a diameter of about 1 nanometer (nm) to about 900 nm, about 50 nm to about 800 nm, about 50 nm to about 700 nm, about 50 nm to about 500 nm, about 50 nm to about 400 nm, about 100 nm to about 300 nm, about 200 nm to about 300 nm, or about 200 nm to about 250 nm. In one or more aspects, the nanoemulsion is passed through a homogenizer to reduce the particle size to nanoscale.

In one or more aspects, the compositions, emulsions, and/or nanoemulsions can include an agent to adjust the pH to a desired level.

In various aspects, the compositions can be present in a pharmaceutical formulation. In one or more aspects, the pharmaceutical composition can be for administration to a subject by any administration route. In certain aspects, the pharmaceutical composition can be suitable for intranasal administration and/or oral administration.

In certain aspects, the pharmaceutical compositions provided herein may further contain buffers and/or pharmaceutically acceptable excipients and/or pharmaceutically acceptable carriers. As is known in the art, pharmaceutically acceptable excipients and/or carriers are relatively inert substances that facilitate administration of a pharmacologically effective substance and can be supplied as liquid solutions or suspensions, as emulsions, or as solid forms suitable for dissolution or suspension in liquid prior to use. The pharmaceutically acceptable carrier may be selected based upon the route of administration desired. For example, an excipient can give form or consistency, or act as a diluent. Suitable excipients include but are not limited to stabilizing agents, wetting and emulsifying agents, salts for varying osmolarity, encapsulating agents, pH buffering substances, and buffers. Suitably the pharmaceutically acceptable carrier helps maintain the construct or viral particle integrity of the viral vector prior to administration, e.g., provide a suitable pH balanced solution. Pharmaceutically acceptable excipients include, but are not limited to, sorbitol, any of the various TWEEN compounds, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like.

The pharmaceutical compositions can be formulated based on the route of administration effective for treating the subject, in various aspects. The exact dosage is chosen by the individual physician in view of the patient to be treated. Dosage and administration are adjusted to provide sufficient levels of the active agent(s) or to maintain the desired effect. Additional factors which are taken into account include the severity of the disease state, e.g., extent of the condition, history of the condition; age, weight and gender of the patient; diet, time and frequency of administration; drug combinations; reaction sensitivities; and tolerance/response to therapy.

Although not required, the compositions may optionally be supplied in unit dosage form suitable for administration of a precise amount, in certain aspects.

### Methods of Treatment

In various aspects, methods are disclosed for treating and/or preventing AD in a subject. The methods can include administering one or more of the compositions disclosed herein to a subject, in various aspects. In certain aspects, the methods can include administering a therapeutically effective amount of one or more of the compositions disclosed herein to a subject in need thereof.

In one or more aspects, methods are disclosed for reducing one or more amyloid beta peptides and/or hyperphosphorylated tau (p-tau) in a subject. The methods can include administering one or more of the compositions disclosed herein to a subject, in various aspects. In certain aspects, the methods can include administering a therapeutically effective amount of one or more of the compositions disclosed herein to a subject in need thereof.

As used herein, “a subject in need thereof” refers to a subject suffering from AD or a subject exhibiting symptoms of AD or suspected of having AD. The term “subject” may be used interchangeably with the terms “individual” and “patient” and includes human and non-human mammalian subjects.

As used herein the term “effective amount” or “therapeutically effective amount” refers to the amount or dose of a therapeutic, including but not limited to one or more of the compositions disclosed herein, and such a dose can include a single or multiple dose administration to the subject, which provides the desired effect. An effective amount can be determined by the attending diagnostician, as one skilled in the art, using known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

In various aspects, an amount of CBD and/or a therapeutically effective amount of CBD in the compositions disclosed herein can be an amount of about 0.05 milligrams (mg)/kilogram (kg) of body weight to about 0.5 mg/kg of body weight, about 0.1 mg/kg of body weight to about 0.4 mg/kg of body weight, about 0.1 mg/kg of body weight to about 0.3 mg/kg of body weight, or about 0.2 mg/kg of body weight.

In various aspects, an amount of melatonin and/or a therapeutically effective amount of melatonin in the compositions disclosed herein can be an amount of about 0.01 milligrams (mg)/kilogram (kg) of body weight to about 0.5 mg/kg of body weight, about 0.02 mg/kg of body weight to about 0.3 mg/kg of body weight, about 0.02 mg/kg of body weight to about 0.1 mg/kg of body weight, or about 0.04 mg/kg of body weight.

In various aspects, an amount of insulin and/or a therapeutically effective amount of insulin in the compositions disclosed herein can be an amount of about 0.001 milligrams (mg)/kilogram (kg) of body weight to about 0.05 mg/kg of body weight, about 0.002 mg/kg of body weight to about 0.02 mg/kg of body weight, about 0.004 mg/kg of body weight to about 0.012 mg/kg of body weight, or about 0.008 mg/kg of body weight. In certain aspects, an amount of insulin and/or a therapeutically effective amount of insulin in the compositions disclosed herein can be an amount of about 1 Unit (U) to about 50 U, about 1 U to about 30 U, about 1 U to about 20 U, or about 1 U to about 10 U, or about 1 U, or about 10 U.

As used herein the term “treating” refers to reducing, eliminating, or improving a condition or disease, or lessening the severity of any aspect of a symptom of a condition or disease. In various aspects, with reference to AD, treating a subject having of diagnosed with AD, suspected of having AD, or at risk of developing AD, can involve reducing, eliminating, or improving one or more symptoms associated with AD. In various aspects, treating AD can include improvements in cognition, memory, and/or mood. In the same or alternative aspects, treating AD can include: 1) reduction of amyloid beta peptides in the brain and/or deposits of amyloid beta peptides in the brain; and/or 2) reduction of hyperphosphorylated tau (p-tau) in the brain and/or deposits of p-tau in the brain.

In some aspects, the compositions or pharmaceutical compositions disclosed herein can be delivered to a subject intranasally. Intranasal administration may be a water-based or emulsion-based delivery system of the compositions or pharmaceutical compositions disclosed herein. In various aspects, the compositions or pharmaceutical compositions disclosed herein may be encapsulated or solubilized for intranasal administration, oral administration, or other route of administration.

In various aspects, the compositions disclosed herein can be administered to a subject on a daily, weekly, and/or monthly basis. In certain aspects, the compositions disclosed herein can be administered to a subject over a period of time. For instance, in one aspect, the compositions can be administered to a subject on a daily basis for a period of one week, two weeks, one month, two months, three months, four months, five months, six months, nine months, or 12 months, or indefinitely as needed.

In certain aspects, the compositions disclosed herein can improve one or more symptoms associated with a subject having AD and/or in a subject suspected of having AD. For instance, in various aspects, the methods disclosed herein can provide improvements in cognition, memory, and/or mood. In certain aspects, the methods disclosed herein may maintain cognition, memory, and/or mood of a subject having AD or suspected of having AD so that such cognition, memory, and/or mood does not deteriorate further. In the same or alternative aspects, the methods disclosed herein can reduce: 1) amyloid beta peptides in the brain and/or deposits of amyloid beta peptides in the brain; and/or 2) hyperphosphorylated tau (p-tau) in the brain and/or deposits of p-tau in the brain. In various aspects, the methods disclosed herein can maintain an amount of amyloid beta peptides and/or deposits thereof in the brain in a subject having AD or suspected of having AD.

### Example 1

Extensive independent research revealed the ability of melatonin and insulin to reverse and prevent Alzheimer's disease (AD)-related cognitive deficits along with Aβ-induced neuroinflammation, mitochondrial impairment, and neuronal cell decline. Other studies have provided evidence that cannabidiol (CBD) has the potential to retard the progression of AD symptoms by reducing Aβ accumulation and tau hyperphosphorylation, making it a valid candidate for AD therapies. In this Example, the ability of CBD-based multi-targeting anti-AD nanoformulations, MIC (melatonin 0.04 mg/kg; insulin 0.008 mg/kg; CBD 0.2 mg/kg) and MC (melatonin 0.04 mg/kg; CBD 0.2 mg/kg), to protect aged APP/PS1 mice from the development of cognitive deficits and AD-related cytopathology was examined. Intranasal administration of MIC and MC once daily for three months improved cognitive functions in APP/PS1 mice significantly. Biochemical studies indicated a significant impact on AD-related neuropathologic changes stimulated by the MC treatment. Results of mitochondrial marker expression suggest that prolonged treatment with MIC and MC nanoformulations balances mitochondrial dynamics and improves mitochondrial activity and mitophagy. Overall, CBD-based combinational therapies have a remarkable neurocognitive effect and a beneficial impact on AD-associated mitochondrial dysfunction, suggesting a promising potential for long-term AD therapy.

### Introduction

Alzheimer's disease (AD) is the most prevalent neurodegenerative disease worldwide, characterized by memory loss and progressive cognitive disability. Although AD is a disease with complex etiology, it has two main hallmarks: extracellular deposits of the amyloid-β peptide (Aβ) and intracellular neurofibrillary tangles (NFT) composed of hyperphosphorylated tau protein (p-tau). These deposits mainly occur in the neocortex, hippocampus, and other subcortical regions of the brain that are pivotal in carrying out cognitive function [1]. While much emphasis has been given to the amyloid and tau proteins, it is evident that AD has a multifactorial etiology. Mitochondrial dysfunction, oxidative stress, insulin intolerance, and neurodegeneration are known to contribute to the pathogenesis of AD.

As of now, treatments targeting a single pathogenic factor of the disease, such as inhibition of Aβ peptide aggregation or tau phosphorylation, have yet to yield satisfactory results. Thus, a treatment that could affect multiple aspects of the disease pathogenesis would be the most beneficial approach in AD therapy [2-5]. In other words, to halt disease progression, medications should interfere with all AD pathogenic events, which are responsible for all the clinical symptoms, including the formation of amyloid plaques and NFT, inflammation, oxidative damage, metabolism dysregulation, etc. Despite the wealth of studies on AD pathology and intense research efforts in the past few decades, no effective therapeutic approach has been translated into clinical application. Therefore, there is an utmost need for the development of new, more effective multitargeting therapeutics that can slow or delay the onset of AD and prevent the progression of the disease by targeting multiple AD-related pathological processes simultaneously [4].

Over the past decade, mounting evidence has indicated that cannabidiol (CBD) and tetrahydrocannabinol (THC), found in marijuana, are useful for the treatment or prevention of AD-related dementia (ADRD) [6-8]. Although these two molecules share the same nature and origin, they have different pharmacological actions in AD therapy. Extensive studies have revealed that low-dose THC has the potency to inhibit Aβ aggregation, restore neuronal connections, and halt the neurodegenerative process, while CBD reduces Aβ production by promoting amyloid precursor protein ubiquitination and inhibiting Aβ-induced tau protein hyperphosphorylation [9-13]. In the past ten years, a number of studies have emerged exploring the effects of low doses of THC. The results indicated that low-dose THC treatment modified brain plasticity and induced long-term behavioral and developmental effects [14]. In turn, CBD has been studied for a significantly longer period and has been found to reduce or even remove the impact of inflammation by reducing oxygen buildup and promoting neuroprotection through different signal transduction pathways mediated indirectly by cannabinoid receptors. CBD has shown promising results in clinical trials to help stop or even reverse the loss of brain function and memory loss in dementia patients [12,15,16]. Since AD is a multifactorial disease, it may be beneficial to develop a multitargeting AD therapy approach that would stimulate a complex cascade of events resulting in AD combinational therapy. Melatonin and insulin were selected as the prospective AD combination therapy agents due to the fact that decreased melatonin secretion and insulin resistance in AD may contribute to the disease's pathophysiology and clinical symptoms [17,18].

Intranasal delivery provides a practical, non-invasive, and efficient method to deliver therapeutic agents to the brain. Prolonged treatment using the intranasal delivery method increases the number of delivered unmetabolized hydrophobic molecules, like THC and CBD, to the brain and thus enhances their therapeutic benefit [15]. Furthermore, drugs administered using the nasal route usually have higher bioavailability, fewer side effects, and result in higher brain exposure at a similar dosage than oral drugs [19,20]. Thus, development of THC and CBD water-based delivery systems is one of the emerging technologies designed to address brain-targeting challenges. Encapsulating or solubilizing the drug is beneficial as it improves bioavailability, distribution, and therapeutic potency [21]. Furthermore, advances in formulation technologies have contributed to the development of a nano-scale delivery system for simultaneous delivery of hydrophobic and hydrophilic molecules to the brain [15]. As a result, THC and CBD nose-to-brain delivery systems were developed via the implementation of a modern nanotechnological high-energy ultrasonication approach.

In this Example, the results of an in vivo study are presented that are focused on the establishment of a therapeutic approach in the treatment of several prominent cytopathologic features found in AD-affected brains, including Aβ plaques, NFT, inflammation, and mitochondrial dysfunction. In the current study, 12 month old APP/PS1 mice received the once-daily intranasal administration of MIC (at the dose of melatonin-0.04 mg/kg, insulin-0.008 mg/kg, and CBD-0.2 mg/kg) or MC (at the dose of melatonin-0.04 mg/kg and CBD-0.2 mg/kg) nanoformulation for 3 months [10]. This study assessed the effects of long-term treatment with MIC and MC nanoemulsions on the advanced stage of AD. The objective of this study was to evaluate the therapeutic potential of prolonged intranasal treatment with either MIC or MC nanoemulsion in AD by examining their modulatory effects on the neuropathologic profile of AD in aged APP/PS1 mice. It is anticipated that targeting multiple AD-related abnormalities with combinational CBD, melatonin, and insulin-containing nasal nanoformulations holds promising therapeutic value for delaying the progression of AD.

### Results

Cognitive function changes initiated by long-term intranasal administration of CBD-based nanoemulsion.

In the current study, APP_{swe}/PS1_{ΔE9} double-transgenic mice derived from the coexpression of mutated APP and PS1 genes were used, which have demonstrated a prematurely accelerated memory decline due to the accumulation of Aβ deposits in the brain as compared with those expressing a single APP or PS1 mutation [22].

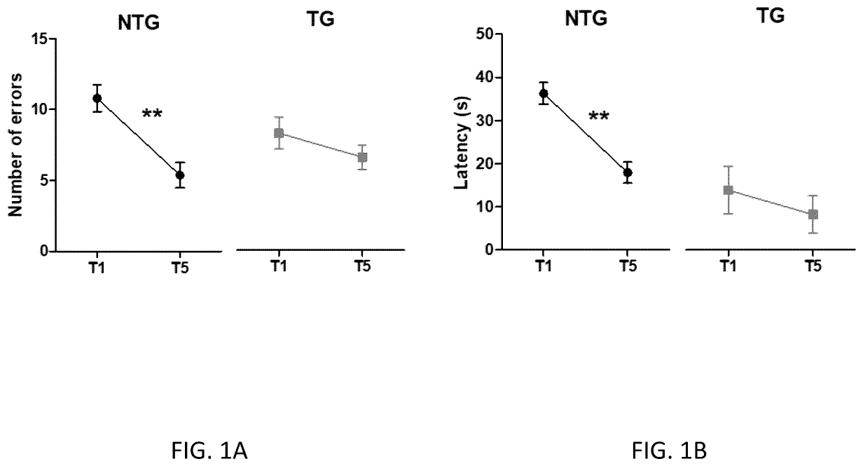

Aged (12-month-old) APP/PS1 mice underwent spatial memory testing on the radial arm water maze (RAWM). Tests were performed before and after the 3-month once-daily intranasal treatment with MIC (melatonin 0.04 mg/kg; insulin 0.008 mg/kg; CBD 0.2 mg/kg) or MC (melatonin 0.04 mg/kg; CBD 0.2 mg/kg) nanoemulsion. Untreated transgenic APP/PS1 mice (TG) and non-transgenic C57BL/6J mice (NTG) were used as controls. The evaluation of the pre-treatment behavior study was conducted for 9 consecutive days. Individual mice underwent five RAWM trials (T) every day. The first four trials were aimed at animal learning and familiarization with the RAWM device, while the very last trial (T5) was the memory test. APP/PS1 mice showed a smaller decrease in the total number of reference errors in RAWM testing than the NTG mice, suggesting impaired short-term spatial memory (FIG. 1A). A significant difference between T1 and T5 latency was found within the NTG group, while the TG group demonstrated no latency difference between their first and last trials (FIG. 1B), which is a sign of memory impairment. The results of pre-intranasal treatment spatial memory evaluation demonstrated that the cognitive performance of the aged NTG control mice was better than that of the aged APP/PS1 mice, as manifested by the relatively rapid learning of a new platform location, a lower number of errors, and less time spent in the wrong place (FIGS. 1A and 1B).

After the pre-treatment spatial memory evaluation, TG mice were divided into control, MIC, and MC-treated groups, so the mean latencies of the study groups were not significantly different. Every mouse from the treatment groups received intranasal administration every day for three months.

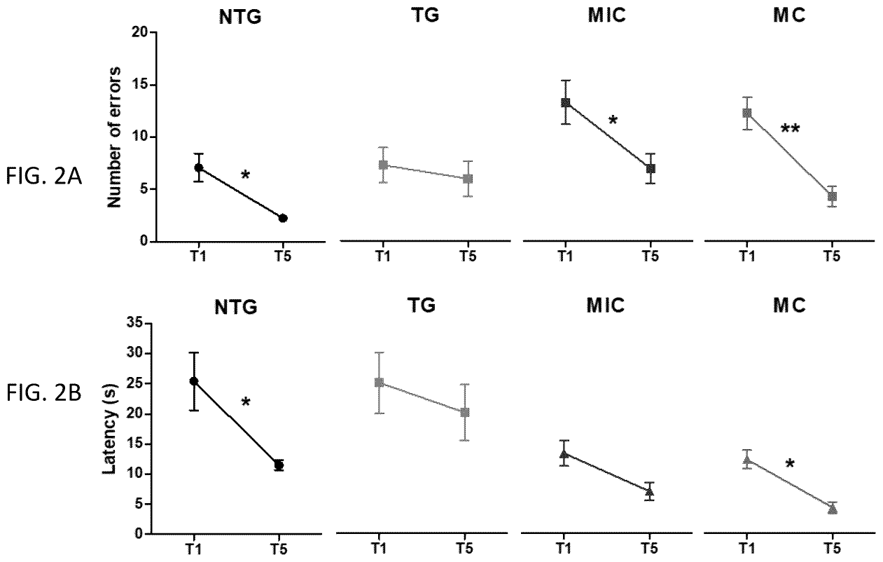

At the end of the 3 months of daily intranasal treatment sessions, the post-treatment RAWM study was conducted for a period of 15 consecutive days. The untreated 15-month-old TG mice demonstrated a significant behavioral impairment in terms of the number of errors made and the time of continuing to make wrong choices by entering the wrong trails (latency in seconds) as compared with the NTG control mice (FIG. 2A and FIG. 2B). In contrast, MIC and MC-treated TG mice showed a significantly lower number of errors in T5 in comparison with T1 than the TG control group (p<0.01 and p<0.001, respectively) (FIG. 2A). Moreover, mice that underwent MC treatment demonstrated a remarkable learning and memory capacity in comparison even with the NTG mice. Overall, TG control mice demonstrated a steady poor spatial memory performance over trials, while APP/PS1 mice treated with MC performed tasks more quickly with a small number of errors and reached a higher level of performance than the normal NTG mouse group. Furthermore, mice that underwent MC treatment demonstrated much higher learning capacity improvements than MIC-treated mice.

The impact of continuous MIC or MC intranasal treatment on the level of Aβ isoforms.

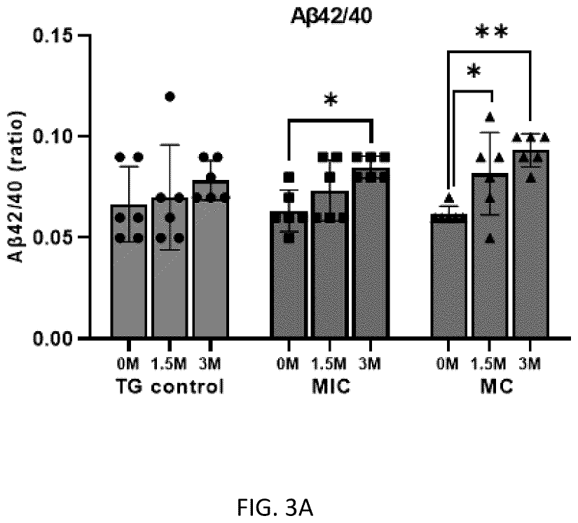

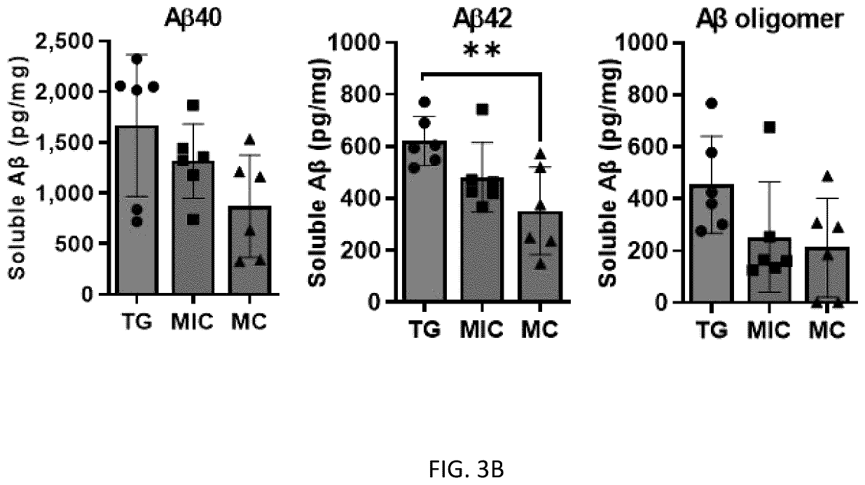

To test the influence of continuous MIC or MC intranasal treatment on Aβ pathology in APP/PS1 mice, a quantitative analysis of Aβ40 and Aβ42 monomers, and oligomer levels in hippocampal brain tissues was conducted using ELISA-based methods. It was discovered that sustained MIC and MC intranasal administration induced a trend toward a decrease in Aβ peptide isoforms and oligomer levels (FIG. 3B). There was a significant reduction in the hippocampal Aβ42 monomer level in the MC-treated group as compared with that in the TG control group (p<0.01). The study of plasma samples revealed a remarkable and steady increase in the Aβ42/40 ratio (p<0.01) in the MC-treated group; however, the MIC group also showed significant improvement in the plasma Aβ42/40 ratio by the end of the study (p<0.05) (FIG. 3A). Therefore, by conducting two separate Aβ evaluation assays, it was confirmed that sustained nose-to-brain delivery of MC nanoformulation significantly reduced Aβ levels in the APP/PS1 mice.

The effect of MIC or MC intranasal treatment on tauopathy in the hippocampus of APP/PS1 mice.

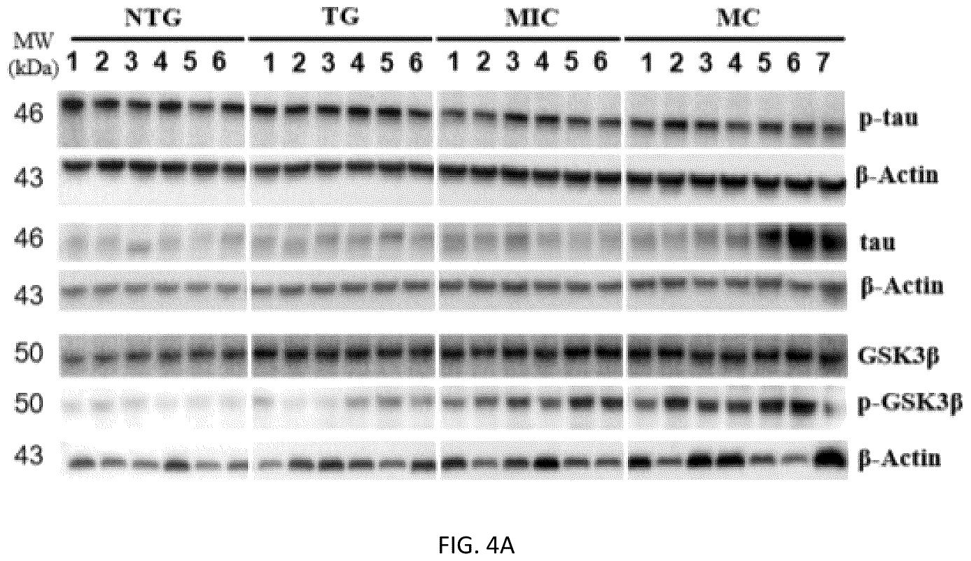

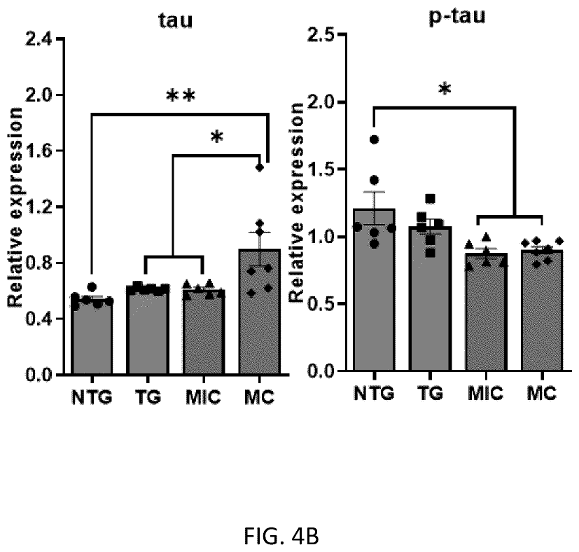

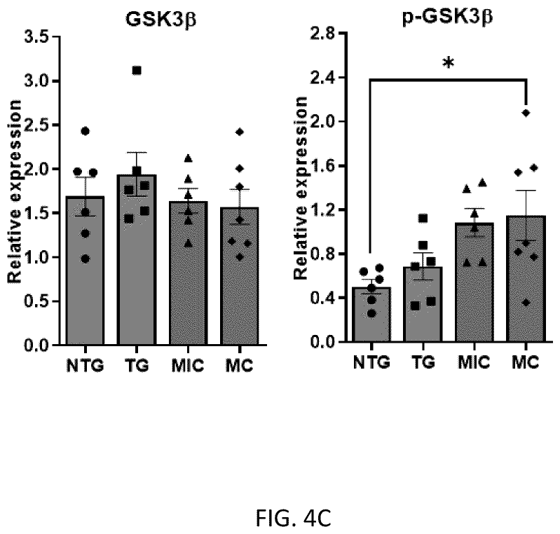

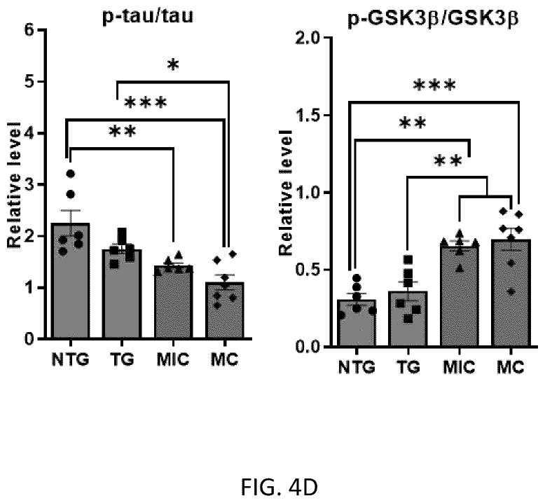

The immunoblotting analysis showed that hippocampal tissue lysate content of p-tau was significantly decreased in both treatment groups compared to the NTG control group (p<0.01); however, no difference was found in comparison to the TG control group (FIG. 4B). A sustained MC intranasal treatment resulted in increased tau expression compared to the NTG and TG controls. The p-tau/tau ratio was lower in the MC group than in the TG group (p<0.001), which was driven by a higher tau level within the treatment group (FIG. 4D). Importantly, continuous intranasal administration of MIC and MC caused a significant increase in the p-GSK3β/GSK3β ratio compared to both controls (FIG. 4D), suggesting the decrease in tau phosphorylation is associated with the decrease in GSK3β activity due to the upregulated phosphorylation of GSK-3β at Ser9 (FIG. 4C) [9,23].

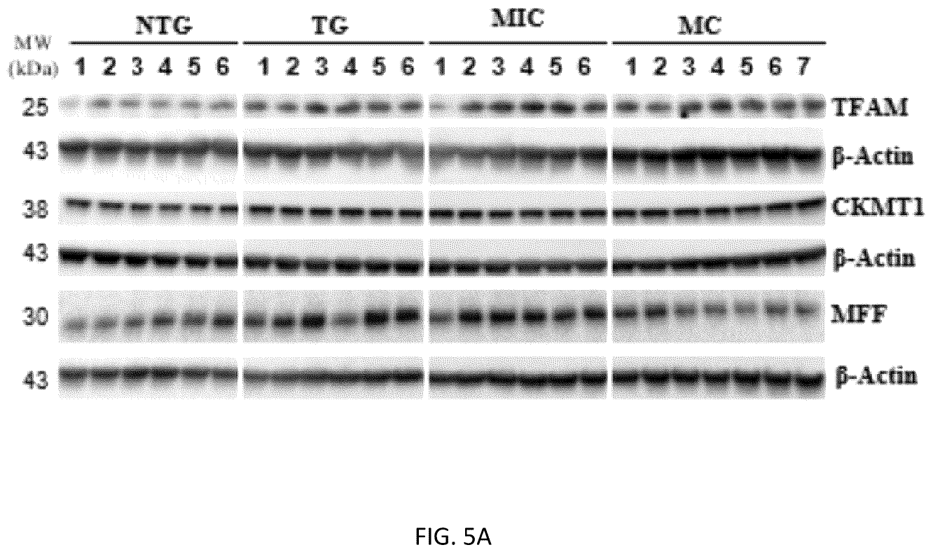

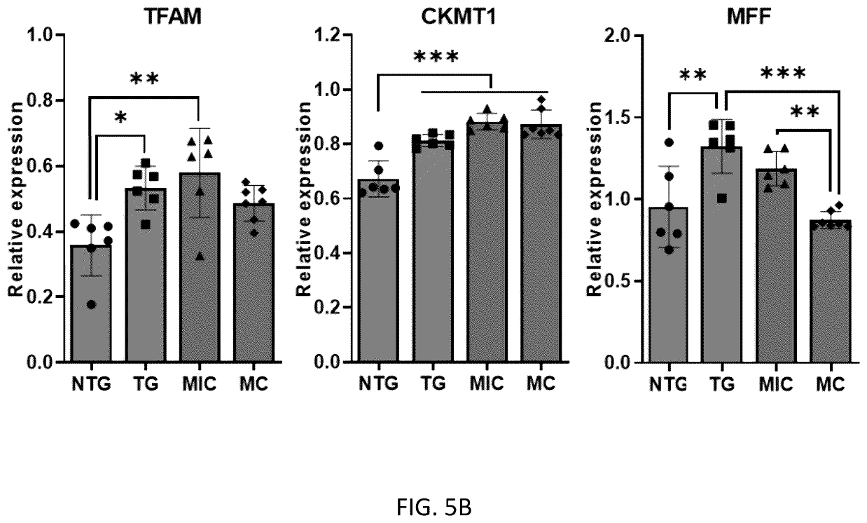

Mitochondrial protein remodeling initiated by prolonged MIC or MC nasal treatment. To evaluate the effect of prolonged intranasal treatment with MIC or MC nanoemulsions on mitochondrial abnormalities in the brains of APP/PS1, Western blot analysis was performed to determine the TFAM, CKMT1, and MFF protein expression levels in the hippocampal tissues (FIGS. 5A-5B). The results revealed a significant increase in the TFAM expression levels in the TG (p<0.05) and MIC-treatment (p<0.001) groups in comparison to the NTG control group. A similar trend was observed in the evaluation of CKMT1 marker expression (p<0.001) which was shown to be steadily overexpressed in all TG groups despite treatment (FIG. 5B). Western blotting revealed upregulated MFF expression in the TG control group. Also, the MFF expression level tended to be higher in the MIC treatment group as compared with the TG control group, although the difference was not statistically significant (p>0.05. FIG. 5B). MC-treated mice, on the other hand, expressed significantly less MFF than the TG control and MIC-treated groups (p<0.001 and p<0.01 respectively). Importantly, no significant difference was found between the NTG control and MC-treatment groups in the expression of TFAM and MFF (FIG. 5B).

After verifying MFF-overexpression in TG control mice by Western blotting, other mitochondrial biomarkers associated with the impairment in mitochondrial content and function induced by MFF-overexpression were investigated. Given the possible changes in mitochondrial fission and fusion balance, the key factors of mitochondrial fission/fusion, and dysfunction were examined. These factors included the fusion proteins: mitofusin-1 (Mfn1), mitofusin-2 (Mfn2), and mitochondrial dynamic like GTPase (Opa1); fission protein: dynamin-1-related protein (Drp1); and mitophagy proteins: PTEN-induced kinase 1 (Pink1) and Parkin.

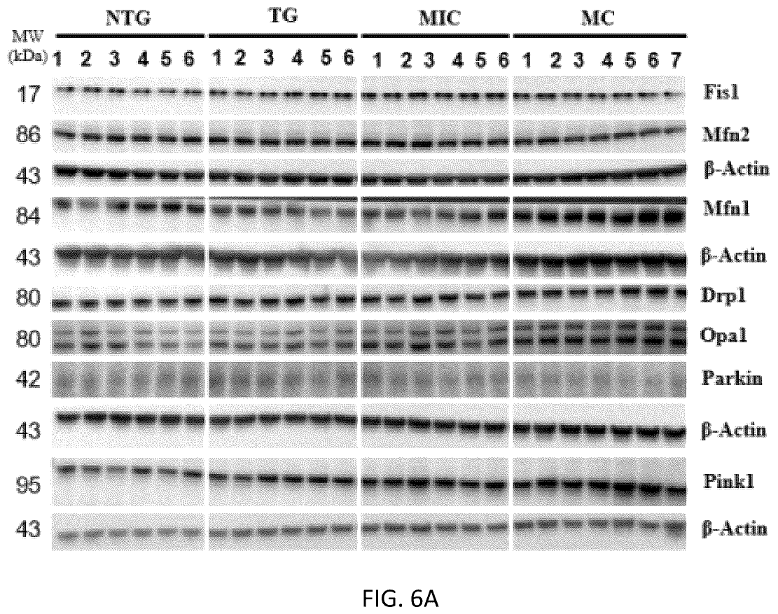

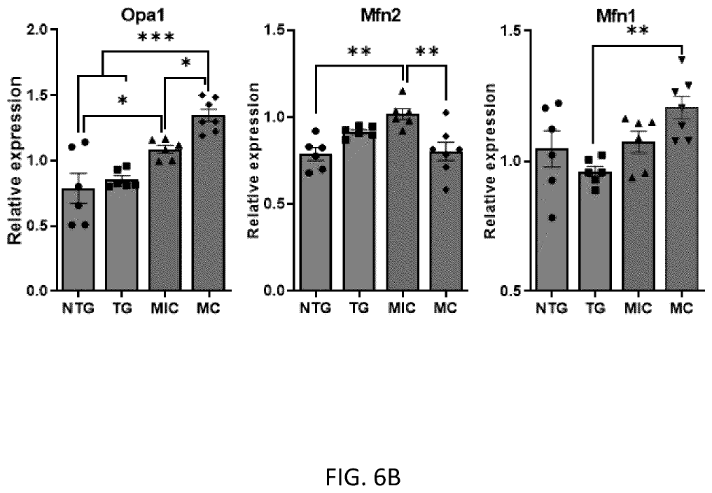

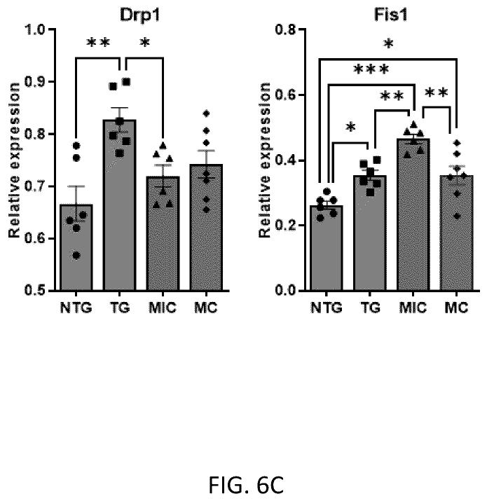

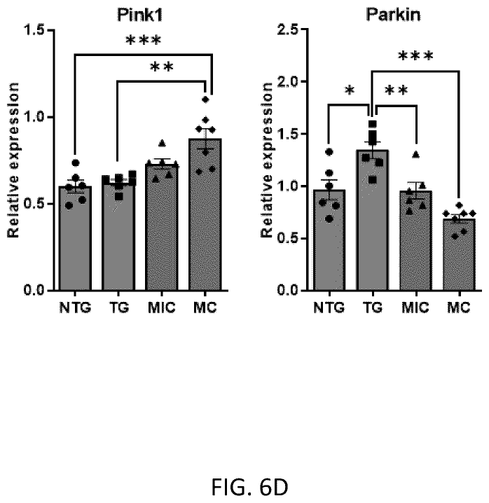

Immunoblot analysis revealed no significant difference in the mitophagy protein Pink1 expression between the NTG and TG control hippocampal tissue samples. In turn, continuous intranasal MC treatment significantly upregulated Pink1 in comparison to the NTG and TG controls (p<0.001 and p<0.01 respectively) (FIG. 6D). Furthermore, TG mice are characterized by enhanced Parkin production, which was significantly decreased after long-term MIC or MC intranasal treatment (p<0.01 and p<0.001 respectively) (FIG. 6D). The TG control group exhibited significantly elevated expression of the fission markers Drp1 and Fis1 (p<0.01 and p<0.05 respectively) (FIG. 6C). These data suggest abnormal mitochondrial distribution in APP/PS1 mouse neurons and an increase in mitophagy pathway activity due to Drp1, Fis1, and Parkin upregulation (FIGS. 6C and 6D). Both treatments (MIC and MC) appeared to normalize the Drp1 expression. However, MIC intranasal administration significantly increased Fis1 expression in TG mouse brains (FIG. 6C). Regarding fusion mitochondrial marker Opa1, Mfn1, and Mfn2 levels, no distinction was detected in TG mice compared with the NTG control mice (FIG. 6B). Intranasal treatment with MIC or MC had a beneficial impact on the expression of all fusion proteins in the APP/PS1 mouse brain tissue (FIG. 6B). In general, both treatments enhanced Opa1 expression levels as compared with the NTG control. Additionally, MC-treated mice expressed a significantly higher level of Mfn2 (p<0.01) protein than TG animals (FIG. 6B). These findings show that prolonged MC intranasal treatment benefits the central nervous system (CNS) by improving mitochondrial movement within neurons and stimulating neuronal cell survival.

The influence of CBD-based nanoemulsion on AD neuropathologic changes in the APP/PS1 mice brain.

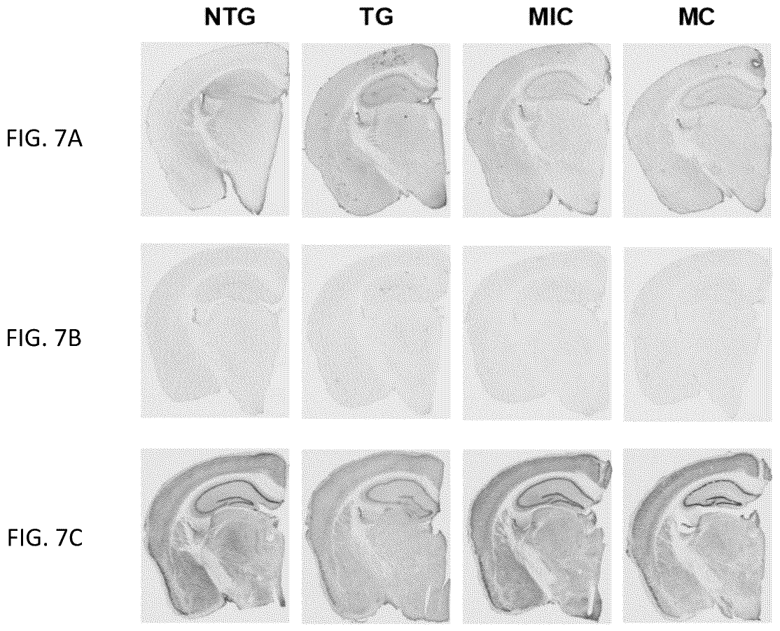

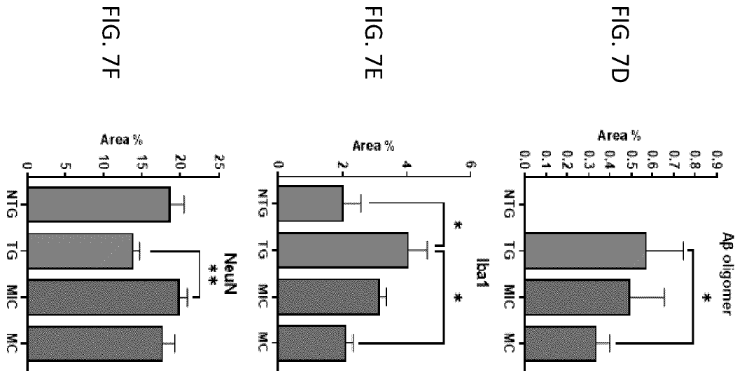

To gain more detailed information about putative neuropathologic changes in the brain tissue of AD-model mice, immunostaining for quantitative analysis of Aβ (FIG. 7A), microglia activation marker-ionized calcium-binding adapter molecule 1 (Iba1) (FIG. 7B), and neuronal nuclei (NeuN) (FIG. 7C) was performed. The immunoblot analysis performed with the anti-Aβ oligomer antibody revealed a significantly lowered Aβ load (plaque density) in the hippocampus of APP/PS1 mice treated with the MC nanoformulation in comparison to the TG control (FIG. 7D). Analysis of acquired images showed increased Iba1 expression in the TG control mice tissue (FIG. 7E). Importantly, MC-treated mice expressed Iba1 at a significantly lower level (p<0.05) than TG control mice, whereas Iba1 expression was not significantly lowered in MIC-treated mice (FIG. 7E). The quantitative immunohistochemical (IHC) data demonstrated statistically significant differences in the percentage of NeuN-positive cells in MIC-treated tissue compared to TG control (p<0.01) (FIG. 7F). MC-treated mice demonstrated increased load with NeuN-positive cells as well; however, this was not significant (FIG. 7F). Taken together, our IHC study data suggest that CBD in combination with melatonin nasal treatment has a beneficial effect on reversing the neuropathologic changes of AD.

### Discussion

The spatial learning capacities of APP/PS1 and C57BL/6J mice before and after treatment were assessed using the classical RAWM test. The pre-treatment RAWM study data demonstrate a statistically significant decline in memory performance of the 12-month-old APP/PS1 mice in comparison to the same-age wild mouse type (FIG. 1A and FIG. 1B). Post-intranasal treatment RAWM data showed that mice treated with MIC or MC intranasally every day for three months exhibited a significantly decreased number of errors compared to the TG control mice's performance. Notably, MC-treated mice demonstrated fewer number of errors than MIC-treated mice and approached NTG mouse parameters. The MC treatment induced a significant reduction in latency values; also, the MC group exhibited shorter latencies in comparison to all animal groups (FIG. 2B). The RAWM memory test clearly showed that intranasal treatment with MIC or MC significantly improved AD-associated cognitive impairments in the aged APP/PS1 mice. The MC treatment, in particular, had an exceptional and maximally beneficial effect on cognition from among the studied nanoformulations.

The substantial memory decline in AD is associated with the increase of Aβ and p-tau deposits. Although many factors contribute to AD pathogenesis, Aβ dyshomeostasis has emerged as the most extensively validated and compelling therapeutic target [24]. Therefore, the expression of several key AD biomarkers in the hippocampal tissue lysates and plasma samples collected from the NTG and TG control mice, MIC-treated TG mice, and MC-treated TG mice were investigated. The results revealed that treatment with the MC formulation significantly increased the Aβ42/40 ratio in the APP/PS1 mouse brains (FIG. 3A), implicating a decrease in Aβ depositions in the brain. The MIC treatment resulted in a significant but relatively less substantive impact compared with the MC treatment. Biochemical analysis of the amyloid peptides isolated from the brain showed that both MIC and MC treatment induced a trend toward a decrease of all studied Aβ isoforms. To illustrate, mice treated with the MC nanoformulation had significantly lower Aβ42 isoform concentration in comparison to control APP/PS1 mice (FIG. 3B). Essentially, the Aβ42 peptide is a putative neurotoxic species that has been shown to disrupt synaptic plasticity and cognition in AD, and can aggregate readily to form Aβ deposits in the brains of AD subjects. Additionally, positive feedback was obtained in quantitative tau and p-tau western blot assays. FIG. 4B indicates that MC treatment stimulated an increase in tau expression, resulting in a significant reduction in the p-tau/tau ratio (FIG. 4D), which is regarded as a robust inhibition of AD progression. The inhibitory effect of MC treatment on the p-tau expression can be attributed to a significant increase in the GSK3β phosphorylation at Ser9 that balances the expression and activation of GSK3β [10].

In order to obtain more evidence of neuropathologic changes in the brain tissue of AD-model control and treated mice, IHC staining for quantitative analysis of Aβ, NeuN, and Iba1 markers was performed (FIGS. 7A-7F). The study proved that MIC and MC intranasal treatments have a noticeable effect on reversing the neuropathologic changes of AD (FIGS. 7A-7F). The MC formulation had a statistically significant Aβ oligomer lowering effect (p<0.05) (FIG. 7A, FIG. 7D). The areas positively stained for NeuN on the hippocampus tissue sections of the control TG and MIC groups were significantly greater than those of the NTG control group (p<0.01) (FIG. 7C, FIG. 7F). Also, stereological counting revealed a diminution in the number of Iba1-positive neurons in the treated mouse brain samples in comparison to TG control animal samples. The MC formulation had the most significant impact on a microglia activation marker in the brain (FIG. 7B, FIG. 7E). It is believed that activated microglia cells, which compose the majority of the inflammatory response, contribute to the neurodegenerative process. Suppression of microglial activation may provide an effective therapeutic intervention that alleviates the progression of neurodegenerative diseases [25,26]. Our IHC study results imply that both MIC and MC nasal treatments improve the neuropathologic changes associated with AD, with the MC formulation having greater anti-AD potential. As follows, multi-targeting MC nanoformulation can attenuate Aβ deposition in hippocampal tissue and its neurotoxic effect, thereby reducing neurodegeneration and rescuing neurons.

Neuronal accumulation of abnormal tau proteins and amyloid plaques are two pathological hallmarks in affected brain regions. Although the detailed mechanism of the pathogenesis of AD is still elusive, a large number of evidence suggests that impaired mitochondria likely play fundamental roles in the pathogenesis of AD. It is believed that a healthy pool of mitochondria not only supports neuronal activity by providing enough energy supply and other related mitochondrial functions to neurons but also guards neurons by minimizing mitochondrial-related oxidative damage [27].

Mitochondrial dysfunction has been established as an early and prominent feature of the disease, suggesting a significant role in the pathogenesis of AD. To obtain preliminary evidence, several main AD-related mitochondrial dysfunction markers: TFAM, CKMT1, and MFF were studied. CKMT1 is a universal and functionally necessary gatekeeper of the permeability transition pore, as its depletion induces mitochondrial depolarization and apoptotic cell death [28]. Neurons display extreme degrees of polarization, so even a slight disruption of CKMT1 could have a devastating impact. All TG groups, despite treatment, overexpress CKMT1 (FIG. 5B) which has been reported to be involved in the adaptation and maintenance of cell viability [29].

The other marker of interest was TFAM, which plays an essential role in the maintenance of mitochondrial homeostasis by regulating its transcription and maintaining mitochondrial DNA (mtDNA) [30]. TFAM is an oxidation biomarker as its upregulation results in an increased mtDNA copy number to protect neurons against mitochondrial oxidative stress [31]. An additional marker of interest was MFF, which is an outer mitochondrial membrane protein that serves as the main molecular mediator for regulating mitochondrial fragmentation [32]. Consequently, it is assumed that the increase of TFAM and MFF production in the TG and MIC groups is related to harmful conditions within the hippocampus. The aforementioned TFAM overexpression (FIG. 5B) has a protective function as it stimulates mitochondrial biogenesis and controls mitochondrial dysfunction under inflammatory, toxic, and oxidative conditions [30]. Additionally, MFF overexpression leads to the enhancement of mitochondrial number by mitochondrial fragmentation in response to acute injury [33].

Furthermore, AD progression is linked to mitochondrial process imbalance, a decrease in mitochondrial function, and neuronal damage in affected brain regions [34-36]. These processes are known as mitochondrial dynamics and consist of fission (the fragmentation of one organelle into two or more, generally aimed at eliminating the altered or dysfunctional portion), fusion (the joining of two or more organelles into one), and mitophagy (degeneration of mitochondria through autophagy) [37]. The fission event of mitochondrial dynamics is regulated by the Drp1 and Fis1 proteins. The fusion process is regulated by Mfn1, Mfn2, and Opa1 [38]. Impaired mitochondria cleaved in the fission process enter the mitophagy stage, which is regulated by Pink1 and Parkin [39]. The balance of fission and fusion mitochondrial markers is required to maintain mitochondrial integrity, biogenesis, cell survival, and stability.

The mitochondrial research data show that MC treatment stimulated Pink1 expression (p<0.01) in comparison to TG control (FIG. 6D). In fact, overexpression of Pink1 ameliorates impaired mitophagy and promotes the removal of damaged mitochondria in Aβ loaded cells, indicating that upregulation of mitophagy factors such as Pink1 may be a potential strategy to treat AD. It was established that mitochondrial fission protein Drp1 expression was reduced by MIC and MC treatment but remained not significantly different from the NTG mouse control; however, both treatments elevated Fis1 levels significantly (FIG. 6C) [40]. The removal of damaged mitochondria through mitophagy tends to be associated with an increase in fission marker levels, as a compensatory mechanism to evade cell death [41].

The findings herein showed that MIC or MC treatment increased the production of mitochondrial fusion markers Opa1, Mfn1, and Mfn2 (FIG. 6B), which initiates another level of quality control by fusion between mitochondria. Thus, the proposed treatment approach rescues two damaged mitochondria by cross-complementation with one another and can mitigate the effects of AD-related damage through the exchange of proteins and lipids with other mitochondria. Mitochondrial fusion can therefore maximize oxidative capacity in response to toxic stress, as long as the stress is below a threshold. Hence, continuous intranasal administration of MIC and MC balances mitochondrial dynamics by mitophagy enrichment with stimulation of compensatory mitochondrial biogenesis by enhancement of fusion/fission events. All this could be an indication of upregulated mitochondrial organelle production, which improves mitochondrial respiration and cellular functions.

Consequently, MC proved to be an anti-AD multitargeting formulation that is able to restore memory deficits in AD transgenic mice by reducing Aβ and p-tau loads, suppressing neurodegeneration, and rebalancing mitochondrial function. In conclusion, MC treatment has a relatively high therapeutic potential for AD due to the synergic beneficial impact of its components on the complex AD pathophysiology, which requires combinational multi-target treatments rather than a single-target treatment.

### Materials and Methods

### Animals

Double transgenic APP_{swe}/PS1_{ΔE9} and C57BL/6J control mice were used in this study. APP_{swe}/PS1_{ΔE9} mice with C57BL/6J background were originally purchased from JAX MMRRC (Stock #034829) and bred in our facility. The APP/PS1 transgenic mice and non-transgenic control mice were maintained at the University of South Florida Department of Comparative Medicine pathogen free animal facility. All mice were acclimated in our vivarium at 20-26° C. at 30-70% humidity with a 12-hour light/dark cycle and were held as one mouse per cage. All APP/PS1 mice were initially genotyped by PCR at the time of grouping and further confirmed by using blood Aβ1-40 measurements. Only the mice expressing Aβ1-40 were used as transgenic mice. Pre-behavior results of RAWM (error and latency) and the plasma Aβ1-40 levels were balanced among TG groups.

All experiments were performed on 12-month-old mice. Twenty-one APP/PS1 mice and eleven C57BL/6J mice were used for behavioral experiments. Prior to the start of treatment with nanoformulation, groups were balanced with respect to gender, plasma Aβ level, body weight, and baseline memory test results within gender. Then, the APP/PS1 mice were divided into three groups: (1) control; (2) MIC; and (3) MC-treated. At the age of 12 months, TG animals were administered intranasal treatment of the MIC (melatonin 0.04 mg/kg; insulin 0.008 mg/kg; CBD 0.2 mg/kg) or MC (melatonin 0.04 mg/kg; CBD 0.2 mg/kg) nanoformulation daily for 3 months. Plasma samples prepared from whole blood were collected at 0, 1.5, and 3 months after beginning the treatment and stored at −80° C. All the animals were euthanized after completing the treatment. Brain tissue samples were collected and stored in liquid nitrogen.

All procedures with animals were conducted in compliance with the National Institutes of Health Guide for Care and Use of Laboratory Animals. All procedures with animals were performed in accordance with the Institutional Animal Care and Use Committee approved protocol (Project ID: IS00000959) and performed according to the National Institutes of Health (NIH) guidelines.

### Materials

CBD (CAS No. 13956-29-1, lot CBD021615-01) was manufactured by Austin Pharma, LLC (Round Rock, TX). Melatonin (Cat. #: PHR1767-500 MG) was purchased from Millipore Sigma (St. Louis, MO, USA) and insulin “Degu” was purchased from Polaris Biology (Chongqing, China). CBD stock solution with a concentration of 5 mg/mL (15.9 mM) was prepared in ethanol and stored in a −20° C. freezer. Lyophilized Aβ1-42 peptide (Catalog No.: 1409-rPEP-02. Biomer Technology, Pleasanton, CA) was suspended in pre-chilled 1,1,1,3,3,3-Hexafluoro-2-propanol (HFIP) on ice to make 1 mM of the Aβ solution. The Aβ solution was stirred at RT for 24 hours until completely dissolved. Then, 10 μl of the Aβ solution was aliquoted into pre-chilled tubes and centrifuged at 1,000×g using Speedvac to evaporate the HFIP. The dried solutes were stored at −80° C. Before usage, Aβ was reconstituted in 1% NH₄OH to 10 mg/mL and diluted into a working solution with 1×Tris-buffered saline (TBS). All other chemicals and solvents were obtained from commercial sources.

### MIC and MC Nanoformulations Preparation

The oil phase, consisting of a mixture of medium-chain triglyceride oil and an emulsifying agent lecithin, was prepared in a beaker. The CBD was dissolved in the oil phase containing an antioxidant, ethanol, and sonicated until the complete dissolution of the cannabinoids. In another beaker, the melatonin and insulin or only melatonin were dissolved in water. To obtain the nanoformulation, the oil was transferred into water phases beaker with an electrical stirrer and sonicator ultrasonic homogenizer for about 10-15 minutes to make an emulsion. The pH of the emulsion was adjusted to somewhere between 6-7. The emulsion was passed through a homogenizer to reduce the particle size to the nanoscale. The particle size was found to be between 200-250 nm.

As a result, the MIC nanoemulsion contained 830 mg/ml CBD, 167 mg/ml melatonin, and 20 U/ml insulin and the MC nanoformulation contained 830 mg/ml CBD and 167 mg/ml melatonin. On a daily basis, a volume of 6 μl was intranasally instilled so each mouse received about 5μ g of CBD, 1μ g of melatonin, and 0.2μ g of insulin. The administered dose of CBD, melatonin, and insulin in the nanoformulations were 0.2, 0.04, and 0.008 mg/kg, respectively.

### Intranasal Administration

MIC and MC were administered intranasally every day for three months. Intranasal administration was performed on mice that were lightly anesthetized with isoflurane. For intranasal administration, we applied a standard method of instillation accepted elsewhere [42]. Each mouse was gently grasped by the back of the neck with the abdomen facing upwards while 6 μl of solution volume was instilled in a nostril.

### Radial Arm Water Maze (RAWM) Behavior Study

The RAWM test was performed to evaluate the effect of MIC and MC nanoemulsions on the spatial learning and memory of APP/PS1 transgenic mice by methods described elsewhere [43,44]. Behavioral assays were carried out during the dark phase under infrared illumination at before and after treatment with nanoformulations or vehicles. Assays were photographed by a video camera, and data were recorded and analyzed using the Bioserve software.

The RAWM device is a black circular water tub with 6 V-shaped stainless-steel structures arranged to form a swimming field with an open central area and 6 arms. Mice were allowed five subsequent trials per day. Every trial (T) started in a different start arm, which could be any of the six-swim paths in the maze (except the goal arm) on a particular day for a particular mouse. The platform location was changed daily to a different arm in a semi-random pattern, and different start arms for each of the daily trials (5 trials) were selected from the remaining five swim arms in a semi-random sequence that involved all arms. On any given day of testing, four acquisition trials (T1-T4) and one retention trial (T5) after a 30-min delay consisted. For any given trial, the number of arm selection errors (number of errors) and the escape latency time (latency) prior to escape onto the platform were recorded. The tested mouse was placed in the starting arm facing the center of the pool for each trial and given 60 seconds to find the platform. Once on the platform, the mouse was allowed to stay there for 30 seconds so that it could observe visual cues before the next trial. If the animal did not find the platform within 1 minute, it was guided, while in the water, toward the platform and left there for 15 seconds. An error was counted each time the mouse entered an arm other than the goal arm. One extra error was recorded when the mouse refused to make at least three choices during that trial. Data from the pre-treatment study that lasted for 9 days was grouped into four (every 3 days of data is presented as one block). And data from the post-treatment study that lasted 15 days was grouped into or five blocks, respectively. Behavior study statistical analysis using ANOVA was performed for the averaged data of T1 vs. T5 of the very last block (block 5).

### Plasma and Tissue Sample Preparation

Whole blood samples were collected before treatment and every 1.5 months from the submandibular vein into an EDTA-containing tube. Tubes were kept on ice and centrifuged at 300×g for 5 minutes, and plasma samples were collected and stored at −80° C. before analysis.

On the day following the last behavioral assay, the animals were anesthetized with SomnaSol (Henry Schein Animal Health. Cat: 024352) and intracardially perfused with 50 ml of saline. Blood samples were taken by intracardial assay and the brain was removed with a sagittal bisection and the left half immersed in freshly prepared 4% paraformaldehyde in PBS (pH 7.4) for histopathology. The rostral portion of this half was processed for biochemical analysis. The right half was dissected and hippocampal tissue was studied. The brain tissue was snap frozen in liquid nitrogen and stored at −80° C.

### Protein Extraction

Frozen brain tissues were thawed and homogenized in the RIPA buffer containing proteinase inhibitor (100 mM Tris, 150 mM NaCl, 0.5% DOC, 1% NP-40, 0.2% SDS, 1 mM Na₃VO₄, 10 mM NaF, 1 mM PMSF, 20 uM Leupeptin) with a pellet pestle motor and 10-second sonication, then centrifuged for 20 min at 21,000×g at 4° C. Crude protein concentrations were determined by Bio-rad DC protein assay (Bio-Rad Cat: 5000112) and adjusted to the same level for all the samples. The supernatants obtained from this protocol were stored at −80° C. The soluble and insoluble Aβ peptide extraction was based on the protocol by Izco M, et al. with a slight modification [45].

### Aβ ELISA Assay

Plasma, hippocampal levels of soluble Aβ40, Aβ42, and oligomer were measured using a commercially available Amyloid beta Human ELISA kit (MegaNanoDiagnostics Inc. Tampa FL, USA) according to manufacturer instructions. Briefly, the brain tissues were homogenized in 400 μl RIPA buffer and sonicated for 20 seconds on ice. Samples underwent centrifugation, and the supernatants were stored at −80° C. Plasma Aβ40 and Aβ42 levels were determined from pre-treatment and during/post-treatment blood samples using the same ELISA kits. Wavelength readings were performed using Synergy H1 Hybrid Multi-Mode Reader (BioTek Instruments, Winooski, Vermont, USA) and corrected by subtracting the readings at 540 nm from the readings at 450 nm. Aβ was quantified using standard curves of purified Aβ40 and Aβ42 length peptides. Oligomeric Aβ was obtained by Aβ42 aggregation.

### Western Blot

Immunoblotting was carried out with the following primary antibodies: tau, p-tau; GSK3β, p-GSK3β, Drp1, Fis1, Opa1, Mfn1, Mfn2, Pink1, Parkin, and β-actin.

Equal amounts of mouse hippocampal protein samples were denatured with a loading buffer (Invitrogen Cat: NP0007) containing 6% β-mercaptoethanol and heated at 70° C. for 10 minutes. Protein samples were then loaded to each well and separated using a 10% Bis-Tris gel. Precision plus protein dual color standards (Bio-rad, #1610374) were used as molecular standards. The separated samples were transferred with wet assay to the PVDF membrane (Millipore Cat: IPFL00010). Membranes were first blocked with 0.2% I-Block buffer for 1 hour at room temperature, and then incubated with the primary antibody at designated dilutions in blocking buffer on a shaker overnight at 4° C. After washing with 1×PBST three times for 5 min, blots were incubated with the appropriate horseradish peroxidase-conjugated secondary antibody in blocking buffer for 1 hour. The enhanced chemiluminescence substrate (Thermo Scientific Prod #34078) was used to develop the blots. The Image J software was used for gel quantification. Primary antibodies used for the protein detection are: p-GSK3β (Cell signaling. Cat: 9336S) (1:2000); GSK3β (Cell signaling. Cat: 9315S) (1:2000); p-tau 217 and tau (MegaNano Diagnostics Inc., Tampa, FL, USA) (1:3000); Drp1 (cell signaling5391) (1:2000); Fis1 (ThermoFisher. Cat: PA5-22142) (1:1000); Opa1 (Proteintech66583) (1:2000); Mfn1 (Proteintech66776) (1:2000); Mfn2 (Proteintech67487) (1:2000); Pink1 (Proteintech23274) (1:2000); Parkin (Proteintech66674) (1:2000) and β-Actin (Sigma Cat: A5441) (1:10000).

### Immunohistochemistry

The left cerebral hemispheres were fixed in 4% PFA, dehydrated through a series of sucrose solutions, and sectioned at 25μ m thickness. The brain sections were incubated overnight at 4° C. with the primary antibodies (1:100 dilution for all) specific to the protein of interest: Aβ A9A (MegaNano Diagnostics Inc.), NeuN (Abcam. Cat: ab3558), and Iba1 (ab178846, Abcam, Cambridge, MA, USA). After incubation with the primary antibody, the brain sections were subjected to an hour incubation with the biotinylated secondary antibody according to the manufacturer's protocol (Vector Laboratories). Light microscopy staining was achieved with the standard biotin-streptavidin/HRP procedure and DAB chromogen. The sections were then counterstained with hematoxylin and mounted under coverslips. For each protein of interest, three sections were selected from the same hippocampus layer of the brain and used for analysis. All three measurements were averaged for each mouse to yield the value for further statistical analysis. The abnormal overstained or cracks were excluded from the analysis field.

### Data Analysis

GraphPad Prism 5.00 software (GraphPad) was used to compare the differences among groups. All results are presented as the mean±standard error of the mean. Comparison of means between more than two independent groups was conducted using one-way ANOVA followed by Tukey's post hoc multiple comparison test. A one-way ANOVA with repeated measures statistical analysis was applied to compare changes in mean scores over time points. Comparison of means between more than three groups was conducted using a regular two-way ANOVA followed by multiple comparison tests. In the case of the use of two-way repeated measures ANOVA multiple comparison test, the p-values were adjusted using the Bonferroni multiple testing correction method.

### REFERENCES FOR EXAMPLE 1

1. Sadigh-Eteghad, S.; Sabermarouf, B.; Majdi, A.; Talebi, M.; Farhoudi, M.; Mahmoudi, J. Amyloid-beta: a crucial factor in Alzheimer's disease. Med Princ Pract 2015, 24, 1-10, doi: 10.1159/000369101.
2. Fakhoury, M. Microglia and Astrocytes in Alzheimer's Disease: Implications for Therapy. Curr Neuropharmacol 2018, 16, 508-518, doi: 10.2174/1570159x15666170720095240.
3. Tondo, G.; Iaccarino, L.; Caminiti, S. P.; Presotto, L.; Santangelo, R.; Iannaccone, S.; Magnani, G.; Perani, D. The combined effects of microglia activation and brain glucose hypometabolism in early-onset Alzheimer's disease. Alzheimers Res Ther 2020, 12, 50, doi: 10.1186/s13195-020-00619-0.
4. Uddin, M. S.; Kabir, M. T.; Jeandet, P.; Mathew, B.; Ashraf, G. M.; Perveen, A.; Bin-Jumah, M. N.; Mousa, S. A.; Abdel-Daim, M. M. Novel Anti-Alzheimer's Therapeutic Molecules Targeting Amyloid Precursor Protein Processing. Oxid Med Cell Longev 2020, 2020, 7039138, doi: 10.1155/2020/7039138.
5. Iqbal, K.; Grundke-Iqbal, I. Alzheimer's disease, a multifactorial disorder seeking multitherapies. Alzheimers Dement 2010, 6, 420-424, doi: 10.1016/j.jalz.2010.04.006.
6. Watt, G.; Karl, T. In vivo Evidence for Therapeutic Properties of Cannabidiol (CBD) for Alzheimer's Disease. Front Pharmacol 2017, 8, 20, doi: 10.3389/fphar.2017.00020.
7. Broers, B.; Patà, Z.; Mina, A.; Wampfler, J.; de Saussure, C.; Pautex, S. Prescription of a THC/CBD-Based Medication to Patients with Dementia: A Pilot Study in Geneva. Med Cannabis Cannabinoids 2019, 2, 56-59, doi: 10.1159/000498924.
8. Nitzan, K.; Ellenbogen, L.; Bentulila, Z.; David, D.; Franko, M.; Break, E. P.; Zoharetz, M.; Shamir, A.; Sarne, Y.; Doron, R. An Ultra-Low Dose of Δ9-Tetrahydrocannabinol Alleviates Alzheimer's Disease-Related Cognitive Impairments and Modulates TrkB Receptor Expression in a 5XFAD Mouse Model. Int J Mol Sci 2022, 23, doi: 10.3390/ijms23169449.
9. Wang, Y.; Hong, Y.; Yan, J.; Brown, B.; Lin, X.; Zhang, X.; Shen, N.; Li, M.; Cai, J.; Gordon, M.; et al. Low-Dose Delta-9-Tetrahydrocannabinol as Beneficial Treatment for Aged APP/PS1 Mice. Int J Mol Sci 2022, 23, doi: 10.3390/ijms23052757.
10. Fihurka, O.; Hong, Y.; Yan, J.; Brown, B.; Lin, X.; Shen, N.; Wang, Y.; Zhao, H.; Gordon, M. N.; Morgan, D.; et al. The Memory Benefit to Aged APP/PS1 Mice from Long-Term Intranasal Treatment of Low-Dose THC. Int J Mol Sci 2022, 23, doi: 10.3390/ijms23084253.
11. Eubanks, L. M.; Rogers, C. J.; Beuscher, A. E. t.; Koob, G. F.; Olson, A. J.; Dickerson, T. J.; Janda, K. D. A molecular link between the active component of marijuana and Alzheimer's disease pathology. Mol Pharm 2006, 3, 773-777, doi: 10.1021/mp060066m.
12. Li, H.; Liu, Y.; Tian, D.; Tian, L.; Ju, X.; Qi, L.; Wang, Y.; Liang, C. Overview of cannabidiol (CBD) and its analogues: Structures, biological activities, and neuroprotective mechanisms in epilepsy and Alzheimer's disease. Eur J Med Chem 2020, 192, 112163, doi: 10.1016/j.ejmech.2020.112163.
13. Calabrese, E. J.; Rubio-Casillas, A. Biphasic effects of THC in memory and cognition. Eur J Clin Invest 2018, 48, e12920, doi: 10.1111/eci.12920.
14. Fishbein, M.; Gov, S.; Assaf, F.; Gafni, M.; Keren, O.; Sarne, Y. Long-term behavioral and biochemical effects of an ultra-low dose of Δ9-tetrahydrocannabinol (THC): neuroprotection and ERK signaling. Exp Brain Res 2012, 221, 437-448, doi: 10.1007/s00221-012-3186-5.
15. Fihurka, O.; Sava, V.; Sanchez-Ramos, J. Dual-function hybrid nanoparticles with gene silencing and anti-inflammatory effects. Nanomedicine (Lond) 2022, doi: 10.2217/nnm-2021-0458.
16. Elsaid, S.; Kloiber, S.; Le Foll, B. Effects of cannabidiol (CBD) in neuropsychiatric disorders: A review of pre-clinical and clinical findings. Prog Mol Biol Transl Sci 2019, 167, 25-75, doi: 10.1016/bs.pmbts.2019.06.005.
17. Cardinali, D. P.; Furio, A. M.; Brusco, L. I. Clinical aspects of melatonin intervention in Alzheimer's disease progression. Curr Neuropharmacol 2010, 8, 218-227, doi: 10.2174/157015910792246209.
18. Watson, G. S.; Craft, S. The role of insulin resistance in the pathogenesis of Alzheimer's disease: implications for treatment. CNS Drugs 2003, 17, 27-45, doi: 10.2165/00023210-200317010-00003.
19. Erdö, F.; Bors, L. A.; Farkas, D.; Bajza, Á.; Gizurarson, S. Evaluation of intranasal delivery route of drug administration for brain targeting. Brain Res Bull 2018, 143, 155-170, doi: 10.1016/j.brainresbull.2018.10.009.
20. Kashyap, K.; Shukla, R. Drug Delivery and Targeting to the Brain Through Nasal Route: Mechanisms, Applications and Challenges. Curr Drug Deliv 2019, 16, 887-901, doi: 10.2174/1567201816666191029122740.
21. Tan, A.; Rao, S.; Prestidge, C. A. Transforming lipid-based oral drug delivery systems into solid dosage forms: an overview of solid carriers, physicochemical properties, and biopharmaceutical performance. Pharm Res 2013, 30, 2993-3017, doi: 10.1007/s11095-013-1107-3.
22. Carrera, I.; Etcheverría, I.; Fernández-Novoa, L.; Lombardi, V.; Cacabelos, R.; Vigo, C. Vaccine Development to Treat Alzheimer's Disease Neuropathology in APP/PS1 Transgenic Mice. Int J Alzheimers Dis 2012, 2012, 376138, doi: 10.1155/2012/376138.
23. Orellana, A. M.; Vasconcelos, A. R.; Leite, J. A.; de Sá Lima, L.; Andreotti, D. Z.; Munhoz, C. D.; Kawamoto, E. M.; Scavone, C. Age-related neuroinflammation and changes in AKT-GSK-3β and WNT/β-CATENIN signaling in rat hippocampus. Aging (Albany NY) 2015, 7, 1094-1111, doi: 10.18632/aging.100853.
24. Selkoe, D. J.; Hardy, J. The amyloid hypothesis of Alzheimer's disease at 25 years. EMBO Mol Med 2016, 8, 595-608, doi: 10.15252/emmm.201606210.
25. Choi, D. K.; Koppula, S.; Suk, K. Inhibitors of microglial neurotoxicity: focus on natural products. Molecules 2011, 16, 1021-1043, doi: 10.3390/molecules16021021.
26. Reale, M.; Conti, L.; Velluto, D. Immune and Inflammatory-Mediated Disorders: From Bench to Bedside. J Immunol Res 2018, 2018, 7197931, doi: 10.1155/2018/7197931.
27. Wang, W.; Zhao, F.; Ma, X.; Perry, G.; Zhu, X. Mitochondria dysfunction in the pathogenesis of Alzheimer's disease: recent advances. Mol Neurodegener 2020, 15, 30, doi: 10.1186/s13024-020-00376-6.
28. Datler, C.; Pazarentzos, E.; Mahul-Mellier, A. L.; Chaisaklert, W.; Hwang, M. S.; Osborne, F.; Grimm, S. CKMT1 regulates the mitochondrial permeability transition pore in a process that provides evidence for alternative forms of the complex. J Cell Sci 2014, 127, 1816-1828, doi: 10.1242/jcs.140467.
29. Enooku, K.; Nakagawa, H.; Soroida, Y.; Ohkawa, R.; Kageyama, Y.; Uranbileg, B.; Watanabe, N.; Tateishi, R.; Yoshida, H.; Koike, K.; et al. Increased serum mitochondrial creatine kinase activity as a risk for hepatocarcinogenesis in chronic hepatitis C patients. Int J Cancer 2014, 135, 871-879, doi: 10.1002/ijc.28720.
30. Aguirre-Rueda, D.; Guerra-Ojeda, S.; Aldasoro, M.; Iradi, A.; Obrador, E.; Ortega, A.; Mauricio, M. D.; Vila, J. M.; Valles, S. L. Astrocytes protect neurons from Aβ1-42 peptide-induced neurotoxicity increasing TFAM and PGC-1 and decreasing PPAR-γ and SIRT-1. Int J Med Sci 2015, 12, 48-56, doi: 10.7150/ijms.10035.
31. Feng, Q.; Shao, M.; Han, J.; Tang, T.; Zhang, Y.; Liu, F. TFAM, a potential oxidative stress biomarker used for monitoring environment pollutants in Musca domestica. Int J Biol Macromol 2020, 155, 524-534, doi: 10.1016/j.ijbiomac.2020.03.208.
32. Sánchez-Alvarez, R.; De Francesco, E. M.; Fiorillo, M.; Sotgia, F.; Lisanti, M. P. Mitochondrial Fission Factor (MFF) Inhibits Mitochondrial Metabolism and Reduces Breast Cancer Stem Cell (CSC) Activity. Front Oncol 2020, 10, 1776, doi: 10.3389/fonc.2020.01776.
33. Otera, H.; Wang, C.; Cleland, M. M.; Setoguchi, K.; Yokota, S.; Youle, R. J.; Mihara, K. Mff is an essential factor for mitochondrial recruitment of Drp1 during mitochondrial fission in mammalian cells. J Cell Biol 2010, 191, 1141-1158, doi: 10.1083/jcb.201007152.
34. Reddy, P. H.; Manczak, M.; Mao, P.; Calkins, M. J.; Reddy, A. P.; Shirendeb, U. Amyloid-beta and mitochondria in aging and Alzheimer's disease: implications for synaptic damage and cognitive decline. J Alzheimers Dis 2010, 20 Suppl 2, S499-512, doi: 10.3233/jad-2010-100504.
35. Chakravorty, A.; Jetto, C. T.; Manjithaya, R. Dysfunctional Mitochondria and Mitophagy as Drivers of Alzheimer's Disease Pathogenesis. Front Aging Neurosci 2019, 11, 311, doi: 10.3389/fnagi.2019.00311.
36. Misrani, A.; Tabassum, S.; Huo, Q.; Tabassum, S.; Jiang, J.; Ahmed, A.; Chen, X.; Zhou, J.; Zhang, J.; Liu, S.; et al. Mitochondrial Deficits With Neural and Social Damage in Early-Stage Alzheimer's Disease Model Mice. Front Aging Neurosci 2021, 13, 748388, doi: 10.3389/fnagi.2021.748388.
37. Guha, S.; Johnson, G. V. W.; Nehrke, K. The Crosstalk Between Pathological Tau Phosphorylation and Mitochondrial Dysfunction as a Key to Understanding and Treating Alzheimer's Disease. Mol Neurobiol 2020, 57, 5103-5120, doi: 10.1007/s12035-020-02084-0.
38. Bertholet, A. M.; Delerue, T.; Millet, A. M.; Moulis, M. F.; David, C.; Daloyau, M.; Arnauné-Pelloquin, L.; Davezac, N.; Mils, V.; Miquel, M. C.; et al. Mitochondrial fusion/fission dynamics in neurodegeneration and neuronal plasticity. Neurobiol Dis 2016, 90, 3-19, doi: 10.1016/j.nbd.2015.10.011.
39. Quinn, P. M. J.; Moreira, P. I.; Ambrósio, A. F.; Alves, C. H. PINK1/PARKIN signalling in neurodegeneration and neuroinflammation. Acta Neuropathol Commun 2020, 8, 189, doi: 10.1186/s40478-020-01062-w.
40. Wang, H.; Zhang, T.; Ge, X.; Chen, J.; Zhao, Y.; Fu, J. Parkin overexpression attenuates Aβ-induced mitochondrial dysfunction in HEK293 cells by restoring impaired mitophagy. Life Sci 2020, 244, 117322, doi: 10.1016/j.1fs.2020.117322.
41. Hernández-Cuervo, H.; Soundararajan, R.; Sidramagowda Patil, S.; Breitzig, M.; Alleyn, M.; Galam, L.; Lockey, R.; Uversky, V. N.; Kolliputi, N. BMI1 Silencing Induces Mitochondrial Dysfunction in Lung Epithelial Cells Exposed to Hyperoxia. Front Physiol 2022, 13, 814510, doi: 10.3389/fphys.2022.814510.
42. Hanson, L. R.; Fine, J. M.; Svitak, A. L.; Faltesek, K. A. Intranasal administration of CNS therapeutics to awake mice. J Vis Exp 2013, doi: 10.3791/4440.
43. Alamed, J.; Wilcock, D. M.; Diamond, D. M.; Gordon, M. N.; Morgan, D. Two-day radial-arm water maze learning and memory task; robust resolution of amyloid-related memory deficits in transgenic mice. Nat Protoc 2006, 1, 1671-1679, doi: 10.1038/nprot.2006.275.
44. Arendash, G. W.; King, D. L.; Gordon, M. N.; Morgan, D.; Hatcher, J. M.; Hope, C. E.; Diamond, D. M. Progressive, age-related behavioral impairments in transgenic mice carrying both mutant amyloid precursor protein and presenilin-1 transgenes. Brain Res 2001, 891, 42-53, doi: 10.1016/s0006-8993 (00) 03186-3.
45. Izco, M.; Pesini, P.; Pérez-Grijalba, V.; Fandos, N.; Sarasa, M. Optimized protocol for amyloid-β extraction from the brain. J Alzheimers Dis 2013, 34, 835-839, doi: 10.3233/jad-121798.

### Numbered Clauses

Clause 1. A method of treating and/or preventing Alzheimer's disease (AD) comprising administering a therapeutically effective amount of a composition to a subject in need thereof, wherein the composition comprises: cannabidiol (CBD) or a pharmaceutically acceptable salt thereof, and melatonin or a pharmaceutically acceptable salt thereof.

Clause 2. The method of clause 1, wherein the composition comprises the cannabidiol (CBD) or a pharmaceutically acceptable salt thereof in an amount of about 0.05 milligrams (mg)/kilogram (kg) of body weight to about 0.5 mg/kg of body weight, about 0.1 mg/kg of body weight to about 0.4 mg/kg of body weight, or about 0.1 mg/kg of body weight to about 0.3 mg/kg of body weight, or about 0.2 mg/kg of body weight.

Clause 3. The method of clause 1 or 2, wherein the composition comprises the melatonin or a pharmaceutically acceptable salt thereof in an amount of about 0.01 milligrams (mg)/kilogram (kg) of body weight to about 0.5 mg/kg of body weight, about 0.02 mg/kg of body weight to about 0.3 mg/kg of body weight, or about 0.02 mg/kg of body weight to about 0.1 mg/kg of body weight, or about 0.04 mg/kg of body weight.

Clause 4. The method of any one of clauses 1-3, wherein the composition further comprises insulin or a pharmaceutically acceptable salt thereof.

Clause 5. The method of clause 4, wherein the composition comprises the insulin or a pharmaceutically acceptable salt thereof in an amount of about 0.001 milligrams (mg)/kilogram (kg) of body weight to about 0.05 mg/kg of body weight, about 0.002 mg/kg of body weight to about 0.02 mg/kg of body weight, or about 0.004 mg/kg of body weight to about 0.012 mg/kg of body weight, or about 0.008 mg/kg of body weight.

Clause 6. The method of any one of clauses 1-5, wherein the composition is an emulsion.

Clause 7. The method of clause 6, wherein the emulsion is a nanoemulsion.

Clause 8. The method of clause 7, wherein the nanoemulsion comprises an oil phase and an aqueous phase.

Clause 9. The method of clause 8, wherein the oil phase and/or the aqueous phase is present as droplets in the nanoemulsion, wherein the droplets have a diameter of about 1 nanometer (nm) to about 900 nm, about 50 nm to about 800 nm, about 50 nm to about 700 nm, about 50 nm to about 500 nm, about 50 nm to about 400 nm, about 100 nm to about 300 nm, about 200 nm to about 300 nm, or about 200 nm to about 250 nm.

Clause 10. The method of any one of clauses 1-9, wherein the composition further comprises one or more oil, one or more aqueous solvent, and/or one or more emulsifier.

Clause 11. The method of clause 10, wherein the composition further comprises one or more oil, and wherein the one or more oil comprises a medium chain triglyceride oil.

Clause 12. The method of clause 10 or 11, wherein the composition further comprises one or more emulsifier, and wherein the one or more emulsifier comprises lecithin.

Clause 13. The method of any one of clauses 1-12, wherein the administering comprises nasal or oral administration.

Clause 14. The method of any one of clauses 1-13, wherein the administering comprises administering the composition to the subject on a daily, weekly, or monthly basis.

Clause 15. The method of any one of clauses 1-14, wherein amyloid beta in the brain of the subject is reduced or eliminated as compared to prior to the administering.

Clause 16. The method of any one of clauses 1-15, wherein amyloid beta 42 peptide and/or amyloid beta 40 peptide in the brain of the subject is reduced or eliminated as compared to prior to the administering.

Clause 17. The method of any one of clauses 1-16, wherein p-tau is reduced or eliminated in the brain of the subject as compared to prior to the administering.

Clause 18. A method of reducing one or more amyloid beta peptides and/or p-tau in the brain of a subject, comprising administering a therapeutically effective amount of a composition to a subject in need thereof, wherein the composition comprises: cannabidiol (CBD) or a pharmaceutically acceptable salt thereof, and melatonin or a pharmaceutically acceptable salt thereof.

Clause 19. The method of clause 18, wherein the composition comprises the cannabidiol (CBD) or a pharmaceutically acceptable salt thereof in an amount of about 0.05 milligrams (mg)/kilogram (kg) of body weight to about 0.5 mg/kg of body weight, about 0.1 mg/kg of body weight to about 0.4 mg/kg of body weight, about 0.1 mg/kg of body weight to about 0.3 mg/kg of body weight, or about 0.2 mg/kg of body weight.

Clause 20. The method of clause 18 or 19, wherein the composition comprises the melatonin or a pharmaceutically acceptable salt thereof in an amount of about 0.01 milligrams (mg)/kilogram (kg) of body weight to about 0.5 mg/kg of body weight, about 0.02 mg/kg of body weight to about 0.3 mg/kg of body weight, about 0.02 mg/kg of body weight to about 0.1 mg/kg of body weight, or about 0.04 mg/kg of body weight.

Clause 21. The method of any one of clauses 18-20, wherein the composition further comprises insulin or a pharmaceutically acceptable salt thereof.

Clause 22. The method of clause 21, wherein the composition comprises the insulin or a pharmaceutically acceptable salt thereof in an amount of about 0.001 milligrams (mg)/kilogram (kg) of body weight to about 0.05 mg/kg of body weight, about 0.002 mg/kg of body weight to about 0.02 mg/kg of body weight, about 0.004 mg/kg of body weight to about 0.012 mg/kg of body weight, or about 0.008 mg/kg of body weight.

Clause 23. The method of any one of clauses 18-22, wherein the composition is an emulsion.

Clause 24. The method of clause 23, wherein the emulsion is a nanoemulsion.

Clause 25. The method of clause 24, wherein the nanoemulsion comprises an oil phase and an aqueous phase.

Clause 26. The method of clause 25, wherein the oil phase and/or the aqueous phase is present as droplets in the nanoemulsion, wherein the droplets have a diameter of about 1 nanometer (nm) to about 900 nm, about 50 nm to about 800 nm, about 50 nm to about 700 nm, about 50 nm to about 500 nm, about 50 nm to about 400 nm, about 100 nm to about 300 nm, about 200 nm to about 300 nm, or about 200 nm to about 250 nm.

Clause 27. The method of any one of clauses 18-26, wherein the composition further comprises one or more oil, one or more aqueous solvent, and/or one or more emulsifier.

Clause 28. The method of clause 27, wherein the composition further comprises one or more oil, and wherein the one or more oil comprises a medium chain triglyceride oil.

Clause 29. The method of clause 27 or 28, wherein the composition further comprises one or more emulsifier, and wherein the one or more emulsifier comprises lecithin.

Clause 30. The method of any one of clauses 18-29, wherein the administering comprises nasal or oral administration.

Clause 31. The method of any one of clauses 18-30, wherein the administering comprises administering the composition to the subject on a daily, weekly, or monthly basis.

Clause 32. The method of any one of clauses 18-31, wherein amyloid beta in the brain of the subject is reduced or eliminated as compared to prior to the administering.

Clause 33. The method of any one of clauses 18-32, wherein amyloid beta 42 peptide and/or amyloid beta 40 peptide in the brain of the subject is reduced or eliminated as compared to prior to the administering.

Clause 34. The method of any one of clauses 18-33, wherein p-tau is reduced or eliminated in the brain of the subject as compared to prior to the administering.

Clause 35. A composition comprising: cannabidiol (CBD) or a pharmaceutically acceptable salt thereof and melatonin or a pharmaceutically acceptable salt thereof.

Clause 36. The composition of clause 35, further comprising an emulsifier, an oil, and/or an aqueous solvent.

Clause 37. The composition of clause 36, wherein the emulsifier comprises lecithin.

Clause 38. The composition of clause 36 or 37, wherein the oil comprises a medium chain triglyceride oil.

Clause 39. The composition of clause 38, wherein the medium chain triglyceride oil comprises a glycerol backbone with an ester linkage to one or more carbon chains comprising about 4 carbon atoms to about 18 carbon atoms, or about 6 carbon atoms to about 12 carbon atoms.

Clause 40. The composition of clause 35, wherein the emulsion is a nanoemulsion.

Clause 41. The composition of clause 40, wherein the nanoemulsion comprises an oil phase and an aqueous phase, and wherein the oil phase and/or the aqueous phase is present as droplets in the nanoemulsion, wherein the droplets have a diameter of about 1 nanometer (nm) to about 900 nm, about 50 nm to about 800 nm, about 50 nm to about 700 nm, about 50 nm to about 500 nm, about 50 nm to about 400 nm, about 100 nm to about 300 nm, about 200 nm to about 300 nm, or about 200 nm to about 250 nm.

Clause 42. The composition of any one of clauses 35-41, wherein the CBD is present in an amount of about 300 milligrams (mg)/milliliter (ml) to about 1500 mg/ml, about 500 milligrams (mg)/milliliter (ml) to about 1200 mg/ml, about 700 milligrams (mg)/milliliter (ml) to about 900 mg/ml, or about 830 mg/ml.

Clause 43. The composition of any one of clauses 35-42, wherein the melatonin is present in an amount of about 10 mg/ml to about 1000 mg/ml, about 50 mg/ml to about 500 mg/ml, about 100 mg/ml to about 200 mg/ml, or about 167 mg/ml.

Clause 44. The composition of any one of clauses 35-43, further comprising insulin or a pharmaceutically acceptable salt thereof.

Clause 45. The composition of clause 44, wherein the insulin is present in an amount of about 1 Unit (U)/ml to about 100 U/ml, about 5 Units (U)/ml to about 70 U/ml, or about 10 Units (U)/ml to about 30 U/ml, or about 20 U/ml.

Clause 46. A pharmaceutical formulation for nasal administration, comprising the composition of any one of clauses 36-45.

Clause 47. A pharmaceutical composition for oral administration, comprising the composition of any one of clauses 36-45.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

All methods described herein can be performed in any suitable order unless otherwise indicated herein or otherwise clearly contradicted by context. The use of any and all examples provided herein, is intended merely to better illuminate the invention and does not pose a limitation on the scope of the invention unless otherwise claimed. No language in the specification should be construed as indicating any non-claimed element as essential to the practice of the invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

What is claimed is:

1. A method of treating and/or preventing Alzheimer's disease (AD) comprising administering a therapeutically effective amount of a composition to a subject in need thereof, wherein the composition comprises:
cannabidiol (CBD) or a pharmaceutically acceptable salt thereof, and melatonin or a pharmaceutically acceptable salt thereof.

2. The method of claim 1, wherein the composition comprises the cannabidiol (CBD) or a pharmaceutically acceptable salt thereof in an amount of about 0.05 milligrams (mg)/kilogram (kg) of body weight to about 0.5 mg/kg of body weight, about 0.1 mg/kg of body weight to about 0.4 mg/kg of body weight, or about 0.1 mg/kg of body weight to about 0.3 mg/kg of body weight, or about 0.2 mg/kg of body weight.

3. The method of claim 1, wherein the composition comprises the melatonin or a pharmaceutically acceptable salt thereof in an amount of about 0.01 milligrams (mg)/kilogram (kg) of body weight to about 0.5 mg/kg of body weight, about 0.02 mg/kg of body weight to about 0.3 mg/kg of body weight, or about 0.02 mg/kg of body weight to about 0.1 mg/kg of body weight, or about 0.04 mg/kg of body weight.

4. The method of claim 1, wherein the composition further comprises insulin or a pharmaceutically acceptable salt thereof.

5. The method of claim 4, wherein the composition comprises the insulin or a pharmaceutically acceptable salt thereof in an amount of about 0.001 milligrams (mg)/kilogram (kg) of body weight to about 0.05 mg/kg of body weight, about 0.002 mg/kg of body weight to about 0.02 mg/kg of body weight, or about 0.004 mg/kg of body weight to about 0.012 mg/kg of body weight, or about 0.008 mg/kg of body weight.

6. The method of claim 1, wherein the composition is an emulsion.

7. The method of claim 6, wherein the emulsion is a nanoemulsion.

8. The method of claim 7, wherein the nanoemulsion comprises an oil phase and an aqueous phase.

9. The method of claim 8, wherein the oil phase and/or the aqueous phase is present as droplets in the nanoemulsion, wherein the droplets have a diameter of about 1 nanometer (nm) to about 900 nm, about 50 nm to about 800 nm, about 50 nm to about 700 nm, about 50 nm to about 500 nm, about 50 nm to about 400 nm, about 100 nm to about 300 nm, about 200 nm to about 300 nm, or about 200 nm to about 250 nm.

10. The method of claim 1, wherein the composition further comprises one or more oil, one or more aqueous solvent, and/or one or more emulsifier.

11. The method of claim 10, wherein the composition further comprises one or more oil, and wherein the one or more oil comprises a medium chain triglyceride oil.

12. The method of claim 10, wherein the composition further comprises one or more emulsifier, and wherein the one or more emulsifier comprises lecithin.

13. The method of claim 1, wherein the administering comprises nasal or oral administration.

14. The method of claim 1, wherein the administering comprises administering the composition to the subject on a daily, weekly, or monthly basis.

15. The method of claim 1, wherein amyloid beta 42 peptide and/or amyloid beta 40 peptide in the brain of the subject is reduced or eliminated as compared to prior to the administering.

16. The method of claim 1, wherein p-tau is reduced or eliminated in the brain of the subject as compared to prior to the administering.

17. A composition in the form of an emulsion comprising:
cannabidiol (CBD) or a pharmaceutically acceptable salt thereof and melatonin or a pharmaceutically acceptable salt thereof.

18. The composition of claim 17, wherein the composition is a nanoemulsion, and wherein the nanoemulsion comprises an oil phase and an aqueous phase, and wherein the oil phase and/or the aqueous phase is present as droplets in the nanoemulsion, wherein the droplets have a diameter of about 1 nanometer (nm) to about 900 nm, about 50 nm to about 800 nm, about 50 nm to about 700 nm, about 50 nm to about 500 nm, about 50 nm to about 400 nm, about 100 nm to about 300 nm, about 200 nm to about 300 nm, or about 200 nm to about 250 nm.

19. The composition of claim 17, wherein the CBD is present in an amount of about 300 milligrams (mg)/milliliter (ml) to about 1500 mg/ml, about 500 milligrams (mg)/milliliter (ml) to about 1200 mg/ml, about 700 milligrams (mg)/milliliter (ml) to about 900 mg/ml, or about 830 mg/ml; and/or wherein the melatonin is present in an amount of about 10 mg/ml to about 1000 mg/ml, about 50 mg/ml to about 500 mg/ml, about 100 mg/ml to about 200 mg/ml, or about 167 mg/ml.

20. The composition of claim 17, further comprising insulin or a pharmaceutically acceptable salt thereof, and wherein the insulin is present in an amount of about 1 Unit (U)/ml to about 100 U/ml, about 5 Units (U)/ml to about 70 U/ml, or about 10 Units (U)/ml to about 30 U/ml, or about 20 U/ml.
